(19) United States
(12) Patent Application Publication
(10) Pub. No.: US 2024/0412816 A1
(43) Pub. Date: Dec. 12, 2024

(54) METHODS AND COMPOSITIONS FOR IMPROVING ACCURACY OF DNA BASED KINSHIP ANALYSIS

(71) Applicant: Verogen, Inc., San Diego, CA (US)

(72) Inventors: June SNEDECOR, San Diego, CA (US); Tim FENNELL, Boulder, CO (US); Seth STADICK, Boulder, CO (US)

(73) Assignee: Verogen, Inc., San Diego, CA (US)

(21) Appl. No.: 18/700,461

(22) PCT Filed: Oct. 12, 2022
(86) PCT No.: PCT/US2022/077984

§ 371 Date: Apr. 11, 2024

Related U.S. Application Data
(60) Provisional application No. 63/255,337, filed on Oct. 13, 2021.

(51) Int. Cl. G16B 20/20; C12Q 1/6876; G16B 40/20

(52) U.S. Cl. CPC G16B 20/20; C12Q 1/6876; G16B 40/20

(57) ABSTRACT

The present disclosure in some aspects relates to improved methods of performing DNA based kinship analysis, including relatives of the first, second, third, fourth, fifth, or sixth degree, or more, including sample preparation, sequencing technologies and methods. In some aspects, the present disclosure describes DNA based kinship analysis that utilizes chromosome-specific kinship probabilities and/or sub-genome kinship coefficients as described herein.

### CROSS-REFERENCE TO RELATED APPLICATIONS

This application claims priority from U.S. provisional application No. 63/255,337 filed Oct. 13, 2021, entitled “METHODS AND COMPOSITIONS FOR IMPROVING ACCURACY OF DNA BASED KINSHIP ANALYSIS,” the contents of which are incorporated by reference in their entirety.

### FIELD

The present disclosure relates in some aspects to methods and compositions for improving accuracy of DNA based kinship analysis in a sample.

### BACKGROUND

Current methods of generating DNA profiles for comparisons in genetic databases include genotyping using dense SNP microarrays and whole genome sequencing (WGS) followed by association of evidentiary samples with distant relatives in databases, which require high quantity and high quality DNA samples, and are not designed for familial searching or forensic purposes. Forensic casework samples are generally low quantity and low quality samples, and data from the current methods requires extensive imputation to generate results capable of being uploaded to a search database. Therefore, there is need for a new and improved method for the generation of DNA based profile analysis.

Moreover, there is a need in genealogy for improving DNA based kinship analysis, particularly with regards to distant relatives, including at the fourth, fifth, and sixth degree, and beyond. There is also a need in genealogy for improving DNA based kinship analysis with regards to more closely related individuals, such as at the first, second, and third degree. Existing approaches to kinship estimation, including the use of whole genome coefficients, have reduced power and higher false positive rates, especially beginning at the fourth degree and increasing exponentially at the fifth degree and beyond, but also at, e.g., the second and third degree. The false positive rate achieved using existing approaches is high enough that when searching a large collection of samples, e.g., for forensic applications, the results are largely unusable.

Segment matching is the gold standard for finding relationships between individuals using SNPs, but it requires many thousands of SNPs to function well. However, for forensics applications, for instance, there is frequently an insufficient amount of DNA to assay the order of magnitude higher number of SNPs needed for applying this approach to identifying distantly related individuals, thereby making it impractical to apply traditional segment matching on these samples. Some existing kinship analyses use fewer SNPs, but do not discriminate well for distant relatives, e.g., of the fourth, fifth, or sixth degree or beyond, thereby leading to false positive results, and does not provide any information about where in the genome two individuals are related.

Therefore, there is a need for a new and improved method for performing a kinship analysis that (a) requires a smaller number of SNPs, (b) reduces false positive rates, particularly among distant relatives of the fourth degree and higher, but also with more closely related relatives of, e.g., the second and third degree, and (c) provides sub-genome granularity as to where in the genome different individuals, including distantly related individuals, share SNPs.

### SUMMARY

Provided herein are methods for performing DNA-based kinship analysis. The methods provided herein provide advantages that include requiring a smaller number of SNPs, reducing false positive rates, particularly among distant relatives of the fourth degree and higher, but also among more closely related relatives of, e.g., the second and third degree, and providing sub-genome granularity as to where in the genome different individuals, including distantly related individuals, share SNPs. Although the methods provided herein are particularly advantageous for more distant relatives, e.g., of the fourth degree and higher, the methods are also effective at reducing the false positive rates among more closely related individuals, including relatives of the third degree.

Accordingly, provided herein is a method for performing DNA-based kinship analysis, comprising: providing a nucleic acid sample; amplifying the nucleic acid sample with a plurality of primers that specifically hybridize to a plurality of target sequences collectively comprising a plurality of single nucleotide polymorphisms (SNPs), thereby generating amplification products; sequencing the amplification products; determining the genotypes of the plurality of SNPs, thereby generating a DNA profile; and calculating the degree of relationship of the DNA profile to a reference DNA profile, wherein the calculating comprises determining a kinship window value for each of a plurality of kinship windows. In some embodiments, the degree of relationship is represented by an overall kinship coefficient for the DNA profile with the reference DNA profile.

In some of any of such embodiments, each of the plurality of kinship windows comprise a different set of SNPs from among the plurality of SNPs. In some of any of such embodiments, each of the plurality of kinship windows corresponds to a continuous segment of a chromosome. In some of any of such embodiments, each of the plurality of kinship windows comprises SNPs that correspond to a continuous segment of a chromosome. In some of any of such embodiments, each of the plurality of kinship windows overlaps with at least one other kinship window from among the plurality of kinship windows. In some of any of such embodiments, the determining the kinship window value for each of the plurality of kinship windows is performed using algorithms and processes of, associated with, or derived from, PC-Relate.

In some of any of such embodiments, the calculating further comprises identifying a group of identified peaks comprising one or more identified peaks across the plurality of kinship windows that exceed a kinship peak threshold value. In some embodiments, the kinship peak threshold value is a value within the range of 0.15 to 0.25.

In some of any of such embodiments, each of the one or more identified peaks comprises a width in centimorgan (cM), and the width for each of the identified peaks is at least the width of a kinship window in cM. In some of any of such embodiments, each of the identified peaks has a width of at least 5, 10, 15, 20, 25, 35, 40, 45, 50, 55, 60, or 65 cM. In some of any of such embodiments, at least one of the identified peaks has a width of at least 20, 21, 22, 23, 24, 25, 26, 27, 28, 29, 30, 31, 32, 33, 34, 35, 36, 37, 38, 39, 40, 41, 42, 43, 44, or 45 cM.

In some of any of such embodiments, the method further comprises excluding from the group of identified peaks any identified peaks that have a width below a minimum peak width. In some embodiments, the minimum peak width is or is about 5, 6, 7, 8, 9, 10, 11, 12, 13, 14, 15, 16, 17, 18, 19, 20, 21, 22, 23, 24, or 25 cM.

In some of any of such embodiments, the method further comprises determining whether one or more of the identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile. In some embodiments, the SNP threshold value is at least 0.90, 0.91, 0.92, 0.93, 0.94, 0.95, 0.96, 0.97, 0.98, or 0.99.

In some of any of such embodiments, each of the identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile. In some embodiments, the SNP threshold value is at least 0.90, 0.91, 0.92, 0.93, 0.94, 0.95, 0.96, 0.97, 0.98, or 0.99.

In some of any of such embodiments, the method further comprises excluding from the group of identified peaks any identified peaks that have a shared SNP fraction value that does not exceed the SNP threshold value from the group of identified peaks.

In some of any of such embodiments, the width in cM of each of the identified peaks in the group of identified peaks is summed to determine the amount of shared DNA with a reference DNA profile. In some embodiments, each of the identified peaks that are summed does not include any identified peak that has a width below a minimum peak width; and/or does not include any identified peak that has a shared SNP fraction value that does not exceed the SNP threshold value. In some embodiments, the minimum peak width is or is about 5, 6, 7, 8, 9, 10, 11, 12, 13, 14, 15, 16, 17, 18, 19, 20, 21, 22, 23, 24, or 25 cM. In some embodiments, the SNP threshold value is at least 0.90, 0.91, 0.92, 0.93, 0.94, 0.95, 0.96, 0.97, 0.98, or 0.99.

In some of any of such embodiments, the calculating comprises determining an overall kinship coefficient, wherein determining the overall kinship coefficient comprises calculating an overall kinship coefficient using the following formula: overall kinship coefficient=[the amount of shared DNA]/4.0/[total amount of genomic DNA]. In some embodiments, the total amount of genomic DNA is the total amount of genomic DNA that was inherited from one parent. In some embodiments, the total amount of genomic DNA is about 3,560 cM.

In some of any of such embodiments, each of the plurality of kinship windows comprises between 25 and 200 SNPs. In some of any of such embodiments, each of the plurality of kinship windows comprises between 75 and 125 SNPs. In some of any of such embodiments, each of the plurality of kinship windows comprises about 60 SNPs or 100 SNPs.

In some of any of such embodiments, each of the plurality of kinship windows comprises a length of between 5 and 70 centimorgan (CM). In some of any of such embodiments, each of the plurality of kinship windows comprises a length of between 20 and 40 cM. In some of any of such embodiments, each of the plurality of kinship windows comprises a length of about 20 cM.

In some of any of such embodiments, each of the plurality of kinship windows comprises between 5 and 70 million base pairs. In some of any of such embodiments, each of the plurality of kinship windows comprises between 20 and 40 million base pairs. In some of any of such embodiments, each of the plurality of kinship windows comprises about 20 million base pairs.

Also provided herein is a method for performing DNA-based kinship analysis, comprising: providing a nucleic acid sample; amplifying the nucleic acid sample with a plurality of primers that specifically hybridize to a plurality of target sequences collectively comprising a plurality of single nucleotide polymorphisms (SNPs), thereby generating amplification products; sequencing the amplification products; determining the genotypes of the plurality of SNPs, thereby generating a DNA profile; and calculating the degree of relationship of the DNA profile to a reference DNA profile, wherein the calculating comprises determining a chromosome-specific kinship value for each of two or more pairs of chromosomes.

In some embodiments, the two or more pairs of chromosomes comprises 10 or more, 11 or more, 12 or more, 13 or more, 14 or more, 15 or more, 16 or more, 17 or more, 18 or more, 19 or more, 20 or more, 21 or more, 22 or more, or 23 pairs of chromosomes. In some of any of such embodiments, the two or more pairs of chromosomes comprises 23 pairs of chromosomes.

In some of any of such embodiments, the calculating comprises determining a total number of shared SNPs between the DNA profile and the reference DNA profile. In some of any of such embodiments, the determining the chromosome specific kinship value is based on a comparison of the shared SNPs between the DNA profile and the reference DNA profile. In some embodiments, the comparison comprises determining a total number of overlapping SNPs between the DNA profile and the reference DNA profile. In some of any of such embodiments, the determining the chromosome specific kinship value for each of the two or more pairs of chromosomes is performed using algorithms and processes of, associated with, or derived from, PC-Relate.

In some of any of such embodiments, the calculating an overall CSKP score for the DNA profile in comparison to the reference DNA profile comprises the use of a random forest model and a chromosome specific kinship value for each of the two or more pairs of chromosomes.

In some embodiments, the generating a CSKP model comprises: (a) calculating a mean and standard deviation of chromosome kinship for each chromosome contained within an unrelated sample training set, wherein the unrelated sample training set comprises samples from unrelated individuals; (b) calculating a z-score for each chromosome kinship; (c) calculating a log survival function on the z-score; (d) calculating a sum of the log survival function for each of the two or more chromosomes; (e) performing a logistic regression on the sum of the log survival function; and (f) training a random forest model on overall kinship, log probability from logistic regression analysis, and total overlapping SNPs between samples.

In some embodiments, the calculating an overall CSKP score comprises: (a) determining a chromosome specific kinship value for each of the two or more pairs of chromosomes and calculating a z-score for each chromosome specific kinship value based on the mean and standard deviation of chromosome kinship for the unrelated sample training set; (b) calculating a log survival function value for the z-score of each chromosome specific kinship value and summing the log survival function values; (c) calculating a log probability using the summed value of log survival function values; and (d) determining an overall CSKP score using the random forest model based on the log probability, the total number of shared SNPs between the DNA profile and the reference DNA profile, and the overall kinship value.

In some of any of such embodiments, the calculating an overall CSKP score for the DNA profile in comparison to the reference DNA profile comprises the use of a random forest model and a chromosome specific kinship value for each of the two or more pairs of chromosomes.

In some embodiments, the calculating an overall CSKP score comprises: (a) determining a chromosome specific kinship value for each of the two or more pairs of chromosomes and calculating a z-score for each chromosome specific kinship value based on a mean and standard deviation of chromosome kinship for an unrelated sample training set, wherein the mean and standard deviation of chromosome kinship for the unrelated sample training set were determined by a model comprising the steps of: (i) calculating a mean and standard deviation of chromosome kinship for each chromosome contained within an unrelated sample training set, wherein the unrelated sample training set comprises samples from unrelated individuals; (ii) calculating a z-score for each chromosome kinship; (iii) calculating a log survival function on the z-score; (iv) calculating a sum of the log survival function for each of the two or more chromosomes; (v) performing a logistic regression on the sum of the log survival function; and (vi) training a random forest model on overall kinship, log probability from logistic regression analysis, and total overlapping SNPs between samples; and (b) calculating a log survival function value for the z-score of each chromosome specific kinship value and summing the log survival function values; (c) calculating a log probability using the summed value of log survival function values; and (d) determining the overall CSKP score using the random forest model based on the log probability, the total number of shared SNPs between the DNA profile and the reference DNA profile, and the overall kinship value. In some of any of such embodiments, the overall CSKP score for the DNA profile each reference DNA profile represents the relatedness of the DNA profile with the reference DNA profile.

In some of any of such embodiments, the plurality of SNPs comprises between 1,000 and 50,000 SNPs. In some of any such embodiments, the plurality of SNPs comprises between 5,000 and 50,000 SNPs. In some of any of such embodiments, the plurality of SNPs comprises between 5,000 and 15,000 SNPs. In some of any of such embodiments, the plurality of SNPs comprises between 9,000 and 11,000 SNPs.

In some of any of such embodiments, the amplification is carried out in one or more multiplex PCR reactions. In some of any of such embodiments, the sequencing is conducted using massively parallel sequencing (MPS). In some of any of such embodiments, the sequencing does not comprise whole genome sequencing (WGS).

In some of any of such embodiments, the nucleic acid sample comprises genomic DNA. In some of any of such embodiments, the nucleic acid sample comprises one or more enzyme inhibitors. In some of any of such embodiments, the one or more enzyme inhibitors comprise one or more inhibitors selected from the group consisting of hematin, humic acid, indigo, and tannic acid.

In some of any of such embodiments, the nucleic acid sample comprises low-quality nucleic acid molecules and/or low quantity nucleic acid molecules. In some embodiments, the low quality nucleic acid molecules are degraded genomic DNA and/or fragmented genomic DNA.

In some of any of such embodiments, the nucleic acid sample is a forensic sample. In some of any of such embodiments, the nucleic acid sample is derived from a buccal swab, paper, fabric, or other substrate that is impregnated with saliva, blood, or other bodily fluid.

In some of any of such embodiments, the nucleic acid sample comprises between or between about 50 pg and 100 ng of genomic DNA. In some of any of such embodiments, the nucleic acid sample comprises between or between about 100 pg and 5 ng of genomic DNA. In some of any of such embodiments, the nucleic acid sample comprises at or about 1 ng of genomic DNA.

In some of any of such embodiments, the plurality of SNPs comprises kinship SNPs. In some of any of such embodiments, the plurality of SNPs comprises kinship SNPs, ancestry SNPs, identity SNPs, phenotype SNPs, X-SNPs, and Y-SNPs. In some of any of such embodiments, the plurality of SNPs comprises SNPs selected from one or more of the groups consisting of kinship SNPs, ancestry SNPs, identity SNPs, phenotype SNPs, X-SNPs, and Y-SNPs. In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

In some of any of such embodiments, the DNA profile in relation to the reference DNA profile is a relative of the first degree, second degree, third degree, fourth degree, fifth degree, sixth degree, or seventh degree.

In some of any of such embodiments, the DNA profile in relation to the reference DNA profile is a relative of the fourth degree or fifth degree.

In some of any of such embodiments, the method further comprises generating a family tree comprising the DNA profile in relation to the reference DNA profile and, optionally, one or more additional reference DNA profiles.

### BRIEF DESCRIPTION OF THE DRAWINGS

FIG. 1 depicts an exemplary schematic of the method of generating a library capable of being sequenced.

FIG. 2 shows the results of the number of loci identified using varying input titrations of genomic DNA, including 5 ng, 2.5 ng, 1 ng, 500 pg, 250 pg, 100 pg, and 50 pg.

FIG. 3 shows the percentage of loci detected (call rate) with degraded DNA using the assay described herein compared to Microarray (GSA) call rate.

FIG. 4 shows the number of loci detected in the presence of the inhibitors hematin, humic acid, indigo, and tannic acid, compared to a reference control.

FIG. 5A shows a receiver operating characteristic (ROC) curve for specificity vs sensitivity that was generated using the chromosome-specific kinship probabilities (CSKP) approach to determining kinship, and FIG. 5B shows a precision-recall curve that was generated using the CSKP approach to determining kinship.

FIG. 6A shows a full ROC curve for kinship by the genome-wide approach, kinship by the CSKP approach, and kinship by the sub-genome approach. The x-axis shows the number of false positive matches returned with the cM≥the threshold. The y-axis shows the number of true positive matches returned with cM≥the threshold. FIG. 6B shows a zoomed in portion of a ROC curve pertaining to the relevant range of thresholds, for kinship by the genome-wide approach, kinship by the CSKP approach, and kinship by the sub-genome approach. The x-axis shows the number of false positive matches returned with the cM≥the threshold. The y-axis shows the number of true positive matches returned with cM≥the threshold. FIG. 6C shows a precision-recall curve for kinship by the genome-wide approach, kinship by the CSKP approach, and kinship by the sub-genome approach. The x-axis shows recall, and the y-axis shows precision. FIG. 6D shows a summary table of the key statistics for the data shown in FIGS. 6A-6C, for each of the three approaches (kinship by the existing genome-wide approach, kinship by the CSKP approach, and kinship by the sub-genome approach).

### DETAILED DESCRIPTION

The practice of the techniques described herein may employ, unless otherwise indicated, conventional techniques and descriptions of molecular biology, cell biology, biochemistry and sequencing technology, which are within the skill of those who practice in the art. Specific illustrations of suitable techniques can be had by reference to the examples herein.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Disclosed herein are methods of performing DNA-based kinship analysis, which include providing a nucleic acid sample, and subsequently amplifying the nucleic acid sample with a plurality of primers that specifically hybridize to a plurality of target sequences collectively comprising a plurality of at least between at or about 1,000 to 50,000 single nucleotide polymorphisms (SNPs), thereby generating amplification products, wherein the amplification is carried out in one or more multiplex PCR reactions. In some embodiments, a nucleic acid library is generated from the amplification products. In some embodiments, the nucleic acid library generated from the amplification products is sequenced, and the genotypes of the plurality of SNPs are determined. In some embodiments, the amplification products are sequenced and amplified, and the genotypes of the plurality of SNPs are determined. In some embodiments, the genotypes of the plurality of SNPs are used to generate a DNA profile. In some embodiments, the degree of relationship of the DNA profile to the reference DNA profile is determined.

Specifically provided herein is a method for performing DNA-based kinship analysis, comprising: providing a nucleic acid sample; amplifying the nucleic acid sample with a plurality of primers that specifically hybridize to a plurality of target sequences collectively comprising a plurality of single nucleotide polymorphisms (SNPs), thereby generating amplification products; sequencing the amplification products; determining the genotypes of the plurality of SNPs, thereby generating a DNA profile; and calculating the degree of relationship of the DNA profile to a reference DNA profile, wherein the calculating comprises determining a kinship window value for each of a plurality of kinship windows.

Also specifically provided herein is a method for performing DNA-based kinship analysis, comprising: providing a nucleic acid sample; amplifying the nucleic acid sample with a plurality of primers that specifically hybridize to a plurality of target sequences collectively comprising a plurality of single nucleotide polymorphisms (SNPs), thereby generating amplification products; sequencing the amplification products; determining the genotypes of the plurality of SNPs, thereby generating a DNA profile; and calculating the degree of relationship of the DNA profile to a reference DNA profile, wherein the calculating comprises determining a chromosome-specific kinship value for each of two or more pairs of chromosomes.

In some embodiments, the methods disclosed herein comprise performing DNA-based kinship analysis, which includes providing a nucleic acid sample, and subsequently amplifying the nucleic acid sample with a plurality of primers that specifically hybridize to a plurality of target sequences collectively comprising a plurality of at least between at or about 5,000 to 50,000 single nucleotide polymorphisms (SNPs), thereby generating amplification products, wherein the amplification is carried out in one or more multiplex PCR reactions. In some embodiments, a nucleic acid library is generated from the amplification products. In some embodiments, the nucleic acid library generated from the amplification products is sequenced, and the genotypes of the plurality of SNPs are determined. In some embodiments, the amplification products are sequenced, and the genotypes of the plurality of SNPs are determined. In some embodiments, the genotypes of the plurality of SNPs are used to generate a DNA profile. In some embodiments, the degree of relationship of the DNA profile to a reference DNA profile is determined, such as by chromosome-specific kinship, such as described in Section V.A., or as determined by sub-genome kinship coefficients, such as described in Section V.B.

In some embodiments, disclosed herein is a plurality of primers that specifically hybridize to a plurality of target sequences collectively comprising at least between at or about 1,000 to 50,000 single nucleotide polymorphisms (SNPs) or at least between at or about 5,000 to 50,000 SNPs in a nucleic acid sample, wherein amplifying the nucleic acid sample using the plurality of primers in one or more multiplex reactions results in amplification products.

In some embodiments, the methods disclosed herein comprise constructing a nucleic acid library, which includes providing a nucleic acid sample, and subsequently amplifying the nucleic acid sample with a plurality of primers that specifically hybridize to a plurality of target sequences collectively comprising a plurality of at least between at or about 1,000 to 50,000 SNPs or at least between at or about 5,000 to 50,000 SNPs, thereby generating amplification products, wherein the amplification is carried out in one or more multiplex PCR reactions. In some embodiments, the amplification products are sequenced, and the genotypes of the plurality of SNPs are determined. In some embodiments, the genotypes of the plurality of SNPs are used to generate a DNA profile.

In some embodiments, the methods disclosed herein comprise constructing a DNA profile, which includes providing a nucleic acid sample, and subsequently amplifying the nucleic acid sample with a plurality of primers that specifically hybridize to a plurality of target sequences collectively comprising a plurality of at least between at or about 1,000 to 50,000 SNPs or at least between at or about 5,000 to 50,000 SNPs, thereby generating amplification products, wherein the amplification is carried out in one or more multiplex PCR reactions. In some embodiments, the amplification products are sequenced, and the genotypes of the plurality of SNPs are determined. In some embodiments, the genotypes of the plurality of SNPs are used to generate a DNA profile.

In some embodiments, the methods described herein comprise identifying genetic relatives of a DNA profile, which includes calculating the degree of relationship of a DNA profile comprising genotypes of at least between at or about 1,000 to 50,000 SNPs or at least between at or about 5,000 to 50,000 SNPs to the a reference DNA profile; and generating a family tree comprising the DNA profile in relation to one or more reference DNA profiles, such as the reference DNA profile.

### III. Samples and Sample Processing

In some aspects, the sample disclosed herein can be or comprise any suitable biological sample, or a sample derived therefrom. In some aspects, the samples described herein are processed and amplified using any known suitable method to complement the methods described herein. Exemplary samples, methods of sample processing and methods of sample amplification are described below.

### A. Nucleic Acid Samples

A nucleic acid sample disclosed herein can be derived from any biological sample. A biological sample may be derived from blood, buccal swabs, hair, teeth, bone, and/or semen. In some embodiments, the biological sample is from a human. In some embodiments, the biological sample is a DNA sample. In some embodiments, the DNA sample is a human DNA sample. In some embodiments, the nucleic acid sample comprises DNA. In some embodiments, the nucleic acid sample comprises human DNA. In some embodiments, the DNA is genomic DNA (gDNA). In some embodiments, the DNA is human genomic DNA (human gDNA). The DNA from which the nucleic acid sample may be obtained may be intact or partially degraded. The DNA from which the nucleic acid sample may be obtained may be compromised, degraded or inhibited due, but not limited to, to source material age, variable extraction, storage procedures or environmental exposure. In some embodiments, the DNA is compromised due to calcium inhibition, cremation, burning, and embalming. In some embodiments, the DNA from which the nucleic acid sample is obtained is a low quantity and/or low quality DNA sample. In some embodiments, the DNA from which the nucleic acid sample is obtained is a low quantity and low quality DNA sample. In some embodiments, the low quality DNA sample comprises low quality nucleic acid molecules. In some embodiments, the low quality nucleic acid molecules are degraded DNA, e.g., genomic DNA, and/or are fragmented DNA, e.g., genomic DNA. In some embodiments, the nucleic acid sample comprises low-quality nucleic acid molecules and/or low quantity nucleic acid molecules. In some embodiments, the nucleic acid sample comprises genomic DNA. In some embodiments, the genomic DNA is human genomic DNA. In some embodiments, the nucleic acid sample comprises genomic DNA derived from a human. In some embodiments, the low quality nucleic acid molecules are degraded genomic DNA and/or fragmented genomic DNA.

In some embodiments, the nucleic acid sample comprises one or more enzyme inhibitors. In some embodiments, the one or more enzyme inhibitors comprise one or more inhibitors selected from the group consisting of hematin, humic acid, indigo, and tannic acid.

In some embodiments, the nucleic acid sample is a forensic sample. In some embodiments, the nucleic acid sample is derived from a buccal swab, paper, fabric, or other substrate that is impregnated with saliva, blood, or other bodily fluid.

In some embodiments, the nucleic acid sample comprises between or between about 50 pg and 100 ng of DNA, e.g., genomic DNA. In some embodiments, the nucleic acid sample comprises between or between about 100 pg and 5 ng of DNA, e.g., genomic DNA. In some embodiments, the nucleic acid sample comprises about 100 pg. 200 pg. 300 pg, 400 pg. 500 pg. 600 pg, 700 pg, 800 pg, 900 pg, 1 ng, 1.25 ng, 1.5 ng. 1.75 ng, 2 ng, 2.25 ng, 2.5 ng. 2.75 ng, 3 ng, 3.25 ng. 3.5 ng. 3.75 ng, 4 ng. 4.25 ng, 4.5 ng, 4.75 ng, or 5 ng of DNA, e.g., genomic DNA, or a value between any two of such values. In some embodiments, the nucleic acid sample comprises at or about 1 ng of DNA, e.g., genomic DNA.

### B. Sample Processing and Amplification

A variety of steps can be performed to prepare or process a nucleic acid sample for and/or during an assay. Except where indicated otherwise, the preparative or processing steps described below can generally be combined in any manner and in any order to appropriately prepare or process a particular sample for analysis and/or sequencing, disclosed herein.

In some embodiments, the amount of the nucleic acid sample provided is, is about, or is less than Ing of genomic DNA. In some embodiments, the methods disclosed herein comprise amplification of the genomic DNA. In some embodiments, amplification of the genomic DNA includes one or more multiplex polymerase chain reactions (PCR) comprising a plurality of primers, thereby generating amplification products. In some embodiments, amplification of the genomic DNA includes a single multiplex PCR reaction. In some embodiments, amplification of the genomic DNA includes two multiplex PCR reactions. In some embodiments, amplification of the genomic DNA includes three multiplex PCR reactions. In some embodiments, amplification of the genomic DNA includes four multiplex PCR reactions. In some embodiments, the amplification is carried out in one or more multiplex PCR reactions, such as one, two, three, or four or more multiplex reactions.

In some embodiments, one or more primers in the plurality of primers are designed in accordance with the atypical design strategy as described in WO 2015/126766 A1, which is hereby incorporated by reference in its entirety. In some embodiments, one or more primers in the plurality of primers is at least 24 nucleotides in length, and/or has a melting temperature that is less than 60 degrees C., and/or is AT-rich with an AT content of at least 60%. In some embodiments, one or more primers in the plurality of primers comprises a length of at least 24 nucleotides that hybridize to the target sequence, and/or has a melting temperature that is between 50 degrees C. and 60 degrees C., and/or is AT-rich with an AT content of at least 60%. In some embodiments, one or more primers in the plurality of primers has a melting temperature that is less than 58 degrees C., or is less than 54 degrees C.

In some embodiments, the genomic DNA may be amplified for a number of cycles using the plurality of primers that hybridize and/or tag a plurality of target sequences collectively comprising a plurality of at least between at or about 5,000 to 50,000 single nucleotide polymorphisms (SNPs). In some embodiments, the plurality of SNPs comprises between 5,000 and 50,000 SNPs, between 5,000 and 15,000 SNPs, or between 9,000 and 11,000 SNPs.

In some embodiments, the genomic DNA may be amplified for a number of cycles using the plurality of primers that hybridize and/or tag a plurality of target sequences collectively comprising at least between at or about 1,000 to 5,000, 10,000, 15,000, 20,000, 25,000, 30,000, 35,000, 40,000, 45,000, or 50,000 SNPs. In some embodiments, the genomic DNA may be amplified for a number of cycles using the plurality of primers that hybridize and/or tag a plurality of target sequences collectively comprising at least between at or about 1,000 to 15,000, 20,000, 25,000, 30,000, 35,000, 40,000, 45,000, or 50,000 SNPs. In some embodiments, the genomic DNA may be amplified for a number of cycles using the plurality of primers that hybridize and/or tag a plurality of target sequences collectively comprising at least between at or about 5,000 to 15,000, 20,000, 25,000, 30,000, 35,000, 40,000, 45,000, or 50,000 SNPs. In some embodiments, the genomic DNA may be amplified for a number of cycles using the plurality of primers that hybridize and/or tag a plurality of target sequences collectively comprising at least between at or about 10,000 to 11,000 SNPs. In some embodiments, the genomic DNA may be amplified for a number of cycles using the plurality of primers that hybridize and/or tag a plurality of target sequences collectively comprising at least between at or about 1,000 to 15,000 SNPs, 2,000 to 15,000 SNPs, 3,000 to 15,000 SNPs, 4,000 to 15,000 SNPs, 5,000 to 15,000 SNPs, 6,000 to 15,000 SNPs, 1,000 to 14,000 SNPs, 2,000 to 14,000 SNPs, 3,000 to 14,000 SNPs, 4,000 to 14,000 SNPs, 5,000 to 14,000 SNPs, 6,000 to 14,000 SNPs, 1,000 to 13,000 SNPs, 2,000 to 13,000 SNPs, 3,000 to 13,000 SNPs, 4,000 to 13,000 SNPs, 5,000 to 13,000 SNPs, 6,000 to 13,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

In some embodiments, the plurality of SNPs comprises at or about 1,000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10,000, 10,100, 10,200, 10,300, 10,400, 10,500, 10,600, 10,700, 10,800, 10,900, 11,000, 11,100, 11,200, 11,300, 11,400, 11,500, 11,600, 11,700, 11,800, 11,900, or 12,000, 12,500 SNPs, 13,000 SNPs, 13,500 SNPs, 14,000 SNPs, 14,500 SNPs, 15,000 SNPs, 15,500 SNPs, 16,000 SNPs, 16,500 SNPs, 17,000 SNPs, 17,500 SNPs, 18,000 SNPs, 18,500 SNPs, 19,000 SNPs, 19,500 SNPs, 20,000, 25,000, 30,000, 35,000, 40,000, 45,000, or 50,000 SNPs.

In some embodiments, the genomic DNA may be amplified for a number of cycles using the plurality of primers that hybridize and/or tag a plurality of target sequences collectively comprising at or about 1,000 SNPs, 1,500 SNPs, 2,000 SNPs, 2,500 SNPs, 3,000 SNPs, 3,500 SNPs, 4,000 SNPs, 4,500 SNPs, 5,000 SNPs, 5,500 SNPs, 6,000 SNPs, 6,500 SNPs, 7,000 SNPs, 7,500 SNPs, 8,000 SNPs, 8,500 SNPs, 9,000 SNPs, 9,500 SNPs, 10,000 SNPs, 10,500 SNPs, 11,000 SNPs, 11,500 SNPs, 12,000 SNPs, 12,500 SNPs, 13,000 SNPs, 13,500 SNPs, 14,000 SNPs, 14,500 SNPs, 15,000 SNPs, 15,500 SNPs, 16,000 SNPs, 16,500 SNPs, 17,000 SNPs, 17,500 SNPs, 18,000 SNPs, 18,500 SNPs, 19,000 SNPs, 19,500 SNPs, or 20,000 SNPs. In some embodiments, the genomic DNA may be amplified for a number of cycles using the plurality of primers that hybridize and/or tag a plurality of target sequences collectively comprising at or about 1,000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, or 12000 SNPs. In some embodiments, the genomic DNA may be amplified for a number of cycles using the plurality of primers that hybridize and/or tag a plurality of target sequences collectively comprising at or about 9,000 to 11,000 SNPs. In some embodiments, the genomic DNA may be amplified for a number of cycles using the plurality of primers that hybridize and/or tag a plurality of target sequences collectively comprising at or about 10,000 SNPs. In some embodiments, the genomic DNA may be amplified for a number of cycles using the plurality of primers that hybridize and/or tag a plurality of target sequences collectively comprising at or about 10,230 SNPs.

In some embodiments, the plurality of SNPs comprises kinship SNPs. In some embodiments, the plurality of SNPs comprises SNPs selected from one or more of the groups consisting of kinship SNPs, ancestry SNPs, identity SNPs, phenotype SNPs, X-SNPs, and Y-SNPs. In some embodiments, the plurality of SNPs comprises kinship SNPs, ancestry SNPs, identity SNPs, phenotype SNPs, X-SNPs, and Y-SNPs. In some embodiments, the plurality of SNPs comprises kinship SNPs.

In some embodiments, the SNPs comprise SNPs that have been filtered with a plurality of genotype samples. In some embodiments, the SNPs are selected from categories including ancestry SNPs, identity SNPs, kinship SNPs, phenotype SNPs, X-SNPs and Y-SNPs. In some embodiments, the ancestry SNPs include between at or about 10-100 SNPs. In some embodiments, the identity SNPs include between at or about 10-200 SNPs. In some embodiments, the kinship SNPs include between at or about 7,000-12,000 SNPs. In some embodiments, the phenotype SNPs include between at or about 1-50 SNPs. In some embodiments, the X-SNPs include between at or about 10-200 SNPs. In some embodiments, the Y-SNPs include between at or about 10-200 SNPs. In some embodiments, the ancestry SNPs include between at or about 0-10% of the total number of SNPs. In some embodiments, the identity SNPs include between at or about 0-10% of the total number of SNPs. In some embodiments, the kinship SNPs include between at or about 80-100% of the total number of SNPs. In some embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs. In some embodiments, 100% of the plurality of SNPs are kinship SNPs. In some embodiments, the phenotype SNPs include between at or about 0-5% of the total number of SNPs. In some embodiments, the X-SNPs include between at or about 0-5% of the total number of SNPs. In some embodiments, the Y-SNPs include between at or about 0-5% of the total number of SNPs. In some embodiments, the SNPs do not include medically informative or minor allele frequency SNPs. A tag region can be any sequence, such as a universal tag region, a capture tag region, an amplification tag region, a sequencing tag region, a UMI tag region, and the like.

In some embodiments, target sequences are purified and enriched, and a library of the original DNA sample, also referred to as a nucleic acid library, is generated. In some embodiments, the purification combines purification beads with an enzyme to purify the amplified targets from other reaction components. In some embodiments, the purified target sequences are enriched by amplification of the DNA and addition of UDI adapters and sequences required for cluster generation. The UDI adapters can tag DNA with a unique combination of sequences that identify each sample for analysis.

In some embodiments, a nucleic acid library is generated from the amplification products, including the amplification products produced by any of the methods or embodiments described herein. As such, in some embodiments, the nucleic acid library comprises the amplification products generated by amplifying the nucleic acid sample with the plurality of primers that specifically hybridize to a plurality of target sequences collectively comprising a plurality of at least between at or about 1,000 to 50,000 SNPs or at least between at or about 5,000 to 50,000 SNPs.

In some embodiments, nucleic acid libraries or DNA libraries are normalized to quantify and check for quality, and pooled by combining equal volumes of normalized libraries to create a pool of libraries capable of being sequenced together on the same flow cell. In some embodiments, the quantification includes the use of a fluorimetric method. In some embodiments, the quantification includes a quantitative PCR method. After the DNA libraries are pooled, they can be denatured and diluted using a sodium hydroxide (NaOH)-based method, and a sequencing control can be added.

In some embodiments, the nucleic acid libraries are quantitated, normalized, denatured and diluted as per instructions given in Forenseq Kintelligence kit User Guide (Verogen PN: V16000120, the contents of which are hereby incorporated by reference in their entirety).

In some embodiments, the nucleic acid libraries of DNA libraries are prepared for sequencing using massively parallel sequencing using any known suitable method to complement the methods described herein.

### III. Sequencing and Analysis

In some aspects, the nucleic acid libraries or DNA libraries described in Section II herein can be sequenced using any known suitable method to complement the methods described herein, and are not limited to any particular sequencing platform. In some aspects, the sample disclosed herein can be analyzed using any known suitable method to complement the methods described herein. Exemplary methods of sequencing and methods analysis are described below.

### A. Sequencing

In some embodiments, the technology for sequencing the nucleic acid libraries or DNA libraries created by practicing the methods described herein comprise the use of polymerase-based sequencing by synthesis, ligation based, pyrosequencing or polymerase-based sequencing methods.

In some embodiments, the nucleic acid library is sequenced as per instructions on MiSeq FGx Sequencing System Reference Guide (document #VD2018006, the contents of which are hereby incorporated by reference in their entirety). In some embodiments, the nucleic acid library that is sequenced as per instructions on MiSeq FGx Sequencing System Reference Guide (document #VD2018006) is denatured.

In some aspects, the sequencing methods disclosed herein comprise the use of massively parallel sequencing (MPS). Accordingly, in some embodiments, the sequencing is conducted using massively parallel sequencing (MPS). In some aspects, the sequencing methods disclosed herein do not comprise the use of whole genome sequencing (WGS). In some aspects, the sequencing methods disclosed herein do not comprise the use of microarrays.

In some embodiments, the sequencing methods disclosed herein detect at or about 90% of the loci of the SNPs.

In some embodiments, the sequencing methods disclosed herein generate an output report comprising the results of the sequencing of the amplification products comprising the plurality of SNPs.

### B. Analysis

In some aspects, the methods disclosed herein involve the use of an analysis module that automatically initiates analysis once the sequencing of the samples (i.e. amplification products) is complete. In some embodiments, the analysis module includes Universal analysis Software (UAS).

In some embodiments, the analysis methods disclosed herein generate an output report comprising the results of the sequencing of the amplification products comprising the plurality of SNPs.

In some embodiments, sequencing results are analyzed using the Forenseq Universal Analysis Software 2.1 (Verogen, San Diego,CA) following the instructions outlined in Forenseq Universal Analysis Software 2.1, and provided in Reference Guide Document #VD2019002, the contents of which are hereby incorporated by reference in their entirety. In some embodiments, sequencing results are analyzed using any subsequent version of the Forenseq Universal Analysis Software 2.1, or using any other available sequence analysis software.

### IV. Genotype and DNA Profile Determination

In some aspects, the output report comprising the results of the sequencing of the amplification products comprising the plurality of SNPs generated by any of the methods described herein can be used to genotype the sample using any known suitable method to complement the methods described herein. In some aspects, the output report comprising the results of the sequencing of the amplification products comprising the plurality of SNPs generated by any of the methods described herein can be used to generate a DNA profile using any known suitable method to complement the methods described herein.

In some embodiments, the DNA profile includes a genotype for each of the plurality of SNPs. In some embodiments, the DNA profile includes a genotype for at least or at least about 80%, 85%, 90%, 91%, 92%, 93%, 94%, 95%, 96%, 97%, 98%, or 99% of the plurality of SNPs. In some embodiments, the DNA profile includes a genotype for at least or at least about 99% or about 100% of the SNPs.

In some embodiments, the DNA profile includes a genotype for each of the plurality of SNPs and the location of the SNP in the genome.

In some embodiments, the methods disclosed herein include determination of hair color, eye color and biogeographical ancestry.

### V. Degree of Relationship Determination

In some aspects, the degree of relationship of the DNA profile described in Section IV herein can be calculated with reference to one or more DNA profiles using any known suitable method to complement the methods described herein.

In some embodiments, the calculating the degree of relationship of the DNA profile to the reference DNA profile comprises determining a chromosome-specific kinship value for each of two or more pairs of chromosomes.

In some embodiments, the calculating the degree of relationship of the DNA profile to a reference DNA profile comprises determining a kinship window value for each of a plurality of kinship windows.

In some embodiments, the calculating the degree of relationship of the DNA profile to a reference DNA profile comprises determining a chromosome-specific kinship value for each of two or more pairs of chromosomes; and comprises determining a kinship window value for each of a plurality of kinship windows.

In some embodiments, the DNA-based kinship analysis described herein includes the use of GEDmatch PRO. In some embodiments, the DNA-based kinship analysis described herein allows for generation of a report with minimal user input. In some embodiments, the DNA-based kinship analysis described herein comprises the use of an algorithm to calculate kinship coefficient. In some embodiments, the kinship coefficient determines the relationship status of the sample or DNA profile to a reference DNA profile on a database. For instance, in some embodiments, the kinship coefficient indicates whether each of the one or more identified genetic relatives is likely to be a great great grandmother, a great great grandfather, a great grandfather, a great grandmother, a grandmother, a grandfather, a first cousin, a first cousin once removed, or a second cousin, based on the relative value of the kinship coefficient. In some embodiments, the reference DNA profiles are part of a genealogy database. As such, the methods provided herein can be repeated using multiple different reference DNA profiles, such as reference DNA profiles that are part of a gencalogy database.

In some embodiments, the DNA-based kinship analysis described herein comprises identifying genetic relatives to at or about the first, second, third, fourth, fifth, sixth, or seventh degree. In some embodiments, the DNA-based kinship analysis described herein comprises identifying genetic relatives to at or about the third, fourth, fifth, sixth, or seventh degree. In some embodiments, the DNA-based kinship analysis described herein comprises identifying genetic relatives to more than the third, fourth, fifth, sixth, or seventh degree. In some embodiments, the DNA-based kinship analysis described herein comprises identifying genetic relatives to the fourth, fifth, or sixth degree. In some embodiments, the DNA profile in relation to the reference DNA profile is a relative of the first degree, second degree, third degree, fourth degree, fifth degree, sixth degree, or seventh degree. In some embodiments, the DNA profile in relation to the reference DNA profile is a relative of the third degree, fourth degree, fifth degree, sixth degree, or seventh degree. In some embodiments, the DNA profile in relation to the reference DNA profile is a relative of the third degree, fourth degree, or fifth degree. In some embodiments, the DNA profile in relation to the reference DNA profile is a relative of the fourth degree, fifth degree, sixth degree, or seventh degree. In some embodiments, the DNA profile in relation to the reference DNA profile is a relative of the fourth degree or fifth degree.

In some embodiments, the DNA-based kinship analysis described herein comprises generating a family tree comprising the DNA profile in relation to one or more DNA profiles.

In some embodiments, the DNA-based kinship analysis described herein comprises identifying suspects through common ancestors.

In some embodiments, methods provided herein further comprise calculating the degree of relationship of the DNA profile to each of one or more additional reference DNA profiles using any of the methods provided herein, i.e., repeating the calculating step with each of one or more additional reference DNA profiles.

In some embodiments, the degree of relationship of the DNA profile to the reference DNA profile is calculated using one or both of (a) chromosome-specific kinship probabilities (CSKP), and/or (b) sub-genome kinship coefficients, in any order. Accordingly, In some embodiments, kinship is determined by one or both of: (a) chromosome-specific kinship probabilities (CSKP), and/or (b) sub-genome kinship coefficients. These approaches are described in detail below, in any order.

### A. Chromosome-Specific Kinship Probabilities (CSKP)

In some embodiments, the calculating the degree of relationship of the DNA profile to the reference DNA profile comprises using chromosome-specific kinship probabilities (CSKP). Accordingly, in some embodiments, the calculating the degree of relationship of the DNA profile to the reference DNA profile comprises determining a chromosome specific kinship value for each of two or more pairs of chromosomes.

The CSKP approach to determining kinship is calculated on a chromosome-by-chromosome basis, and provides a probability that kinship between two individuals is true.

In some embodiments, the two or more pairs of chromosomes comprises 10 or more, 11 or more, 12 or more, 13 or more, 14 or more, 15 or more, 16 or more, 17 or more, 18 or more, 19 or more, 20 or more, 21 or more, 22 or more, or 23 pairs of chromosomes. In some embodiments, the two or more pairs of chromosomes is 2, 3, 4, 5, 6, 7, 8, 9, 10, 11, 12, 13, 14, 15, 16, 17, 18, 19, 20, 21, 22, or 23 pairs of chromosomes. The two or more pairs of chromosomes can, in some embodiments, be any two or more pairs of chromosomes selected from among the 23 pairs of chromosomes in a human genome, i.e., two or more pairs selected from the group consisting of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, and the pair of sex chromosomes (chromosomes X and X (X/X), or chromosomes X and Y (X/Y)). In some embodiments, the two or more pairs of chromosomes comprises any two or more pairs of chromosomes selected from the group consisting of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, and chromosome 22. In some embodiments, the two or more pairs of chromosomes comprises 22 pairs of chromosomes. In some embodiments, the 22 pairs of chromosomes comprises chromosome numbers 1 through 22. In some embodiments, the two or more pairs of chromosomes does not comprise sex chromosomes (X and/or Y). In some embodiments, the two or more pairs of chromosomes comprises 23 pairs of chromosomes.

In some embodiments, the calculating the degree of relationship of the DNA profile to the reference DNA profile comprises determining a total number of shared SNPs between the DNA profile and the reference DNA profile.

In some embodiments, the determining the chromosome specific kinship value is based on a comparison of the shared SNPs between the DNA profile and the reference DNA profile, for each chromosome. Accordingly, in some embodiments, the calculating the degree of relationship of the DNA profile to the reference DNA profile comprises determining a chromosome specific kinship value for each chromosome based on a comparison of the shared SNPs between the DNA profile and the reference DNA profile. In some embodiments, the comparison comprises determining a total number of overlapping SNPs between the DNA profile and the reference DNA profile, among all of the two or more pairs of chromosomes, such as among all 23 pairs of chromosomes, or among chromosomes 1 through 22, or among any combination of the 23 pairs of chromosomes. In some embodiments, the determining the chromosome specific kinship value for each of the two or more pairs of chromosomes is performed using algorithms and processes of, associated with, or derived from, PC-Relate. In some embodiments, the determining the chromosome specific kinship value for each of the two or more pairs of chromosomes is performed in accordance with algorithms and/or processes from PC-Relate.

In some embodiments, the calculating the degree of relationship of the DNA profile to the reference DNA profile comprises generating a CSKP model and calculating an overall CSKP score for the DNA profile in comparison to the reference DNA profile. In some embodiments, the CSKP model comprises the use of a random forest model.

In some embodiments, the generating a CSKP model comprises: (a) calculating a mean and standard deviation of chromosome kinship for each chromosome contained within an unrelated sample training set, wherein the unrelated sample training set comprises samples from unrelated individuals; (b) calculating a z-score for each chromosome kinship; (c) calculating a log survival function on the z-score; (d) calculating a sum of the log survival function for each of the two or more chromosomes; (c) performing a logistic regression on the sum of the log survival function; and (f) training a random forest model on overall kinship, log probability from logistic regression analysis, and total overlapping SNPs between samples. The calculations used in generating the CSKP model can be performed using methods known in the art.

In some embodiments, the calculating an overall CSKP score comprises: (a) determining a chromosome specific kinship value for each of the two or more pairs of chromosomes and calculating a z-score for each value based on the mean and standard deviation of chromosome kinship for the unrelated sample training set; (b) calculating a log survival function value for the z-score of each chromosome specific kinship value and summing the log survival function values; (c) calculating a log probability using the summed value of log survival function values; and (d) determining an overall CSKP score using the random forest model based on the log probability, the total number of shared SNPs between the DNA profile and the reference DNA profile, and the overall kinship value.

In some embodiments, the determining the chromosome specific kinship value for each of the two or more pairs of chromosomes is performed using algorithms and processes of, associated with, or derived from, PC-Relate.

In some embodiments, the calculating an overall CSKP score comprises: (a) determining a chromosome specific kinship value for each of the two or more pairs of chromosomes and calculating a z-score for each chromosome specific kinship value based on a mean and standard deviation of chromosome kinship for an unrelated sample training set, wherein the mean and standard deviation of chromosome kinship for the unrelated sample training set were determined by a CSKP model comprising the steps of: (i) calculating a mean and standard deviation of chromosome kinship for each chromosome contained within an unrelated sample training set, wherein the unrelated sample training set comprises samples from unrelated individuals; (ii) calculating a z-score for each chromosome kinship; (iii) calculating a log survival function on the z-score; (iv) calculating a sum of the log survival function for each of the two or more chromosomes; (v) performing a logistic regression on the sum of the log survival function; and (vi) training a random forest model on overall kinship, log probability from logistic regression analysis, and total overlapping SNPs between samples; and (b) calculating a log survival function value for the z-score of each chromosome specific kinship value and summing the log survival function values; (c) calculating a log probability using the summed value of log survival function values; and (d) determining the overall CSKP score using the random forest model based on the log probability, the total number of shared SNPs between the DNA profile and the reference DNA profile, and the overall kinship value. In some embodiments, the overall CSKP score for the DNA profile with each reference DNA profile represents the relatedness of the DNA profile with the reference DNA profile.

In some embodiments, the calculating the degree of relationship of the DNA profile to the reference DNA profile comprises calculating an overall CSKP score for the DNA profile in comparison to the reference DNA profile, in accordance with any of the methods provided herein. In some embodiments, the calculating the degree of relationship of the DNA profile to the reference DNA profile can be used to improve the identification of relatedness for individuals of the first, second, third, fourth, fifth, sixth, or seventh degree or higher.

### B. Sub-Genome Kinship Coefficients

In some embodiments, the calculating the degree of relationship of the DNA profile to the reference DNA profile comprises determining a kinship window value for each of a plurality of kinship windows.

Kinship coefficients are typically calculated on a genome wide scale. However, it is known that DNA is inherited in large segments that are reduced over generations by cross-over during meiosis. For instance, when there is a small amount of shared DNA, e.g., 2%, that is shared between two individuals, the expectation is that the shared DNA, e.g., 2%, is clustered together into a small number of segments of the genome, rather than being distributed evenly throughout the genome. Accordingly, by calculating “sub-genome” kinship coefficients, the kinship of more distant relatives, e.g., of the fourth, fifth, or sixth degree, can be more effectively filtered and determined, and this can also provide more information about where specifically within the genome two individuals are related, i.e., what chromosomes and segments of those chromosomes share DNA between the two individuals. Moreover, this approach can also be taken with more closely related individuals, e.g., of the first, second, or third degree, to reduce the rate of false positives and to provide information about where specifically within the genome two individuals are related. In some embodiments, the same calculations used in the art for calculating genome-wide kinship coefficients, e.g., calculations used in the PC-Relate method, are used for calculating each of the sub-genome kinship coefficients that are region-specific, which are then combined to determine kinship using the methods described herein.

The sub-genome kinship coefficient approach described herein generates a series of kinship values (also referred to as kinship window values) based on a subset of SNPs from the total set of SNPs used across the genome that are contained within each of a plurality of kinship windows, and then those kinship window values are combined in order to give region-specific “hot spots” of similarity. For instance, in cases where overall kinship is low, such as with distant relatives, e.g., of the fourth, fifth, or sixth degree, a sub-genome kinship coefficient can be calculated on a sliding window basis over each chromosome (and thus the genome) to get an estimate of local kinship, such as by having kinship windows overlap across each chromosome.

Conceptually, correct values for kinship at a single SNP, and thereby for small regions of chromosomes, are: 0 (if neither of the two chromosomes is shared between the two individuals), 0.25 (if one of the two chromosomes is shared between the two individuals), or 0.5 (if both of the two chromosomes is shared between the two individuals). This is in contrast to a genome-wide kinship coefficient that may determine kinship using any real value between, for instance, 0 and 0.5, but runs the risk of being a false positive for reasons discussed above.

The expectation is that for two samples that are truly unrelated, i.e., a true negative, the kinship coefficient should always be zero (0), but, due to noise, imperfect models, and other sources of error, a local kinship coefficient may reach values significantly higher than zero, but should rarely peak near 0.25. In contrast, it is expected that samples from two individuals who are distantly related (e.g., of the fourth, fifth, or sixth degree) would mirror the pattern of kinship of unrelated samples in regions of the genome where they share no DNA by inheritance, i.e., have a kinship coefficient of zero (0), and to have a kinship coefficient peaking up to 0.25 within regions of the genome where shared DNA by inheritance is present. The sub-genome kinship coefficient approach provides a method for identifying these regions of the genome where shared DNA by inheritance is present.

Accordingly, in some embodiments, the calculating the degree of relationship of the DNA profile to the reference DNA profile comprises using sub-genome kinship coefficients. In some embodiments, the calculating the degree of relationship of the DNA profile to the reference DNA profile comprises determining a kinship window value for each of a plurality of kinship windows. Thus, in some embodiments, the methods provided herein use sub-genome kinship coefficients, also referred to as sub-genome coefficients, to determine overall kinship, which is particularly advantageous when determining relatedness among more distant relatives, e.g., of the fourth, fifth, sixth, or seventh degree, but is also advantageous when determining relatedness among more closely related individuals, e.g., of the first, second, or third degree.

In some embodiments, the degree of relationship is represented by an overall kinship coefficient for the DNA profile with the reference DNA profile. Thus, in some embodiments, the calculating the degree of relationship of the DNA profile to the reference DNA profile comprises calculating an overall kinship coefficient for the DNA profile with the reference DNA profile. The overall kinship coefficient for the DNA profile represents the relatedness of the DNA profile with the reference DNA profile, i.e., the overall kinship coefficient is a measure of relatedness between the DNA profile and the reference DNA profile. For instance, an overall kinship coefficient of 0.25 is expected for a sibling relationship or a parent-offspring relationship, whereas an overall kinship coefficient of 0.125 would be expected for a grandparent-grandchild relationship, and an overall kinship coefficient of 0.0625 would be expected for a first cousin (fourth degree) relationship, and an overall kinship coefficient of 0.03125 would be expected for a second cousin (fifth degree) relationship. The overall kinship coefficient can be calculated in accordance with the methods described herein. In some embodiments, the degree of relationship of the DNA profile to the reference DNA profile is represented by an overall kinship coefficient for the DNA profile with the reference DNA profile.

In some embodiments, a sub-genome kinship coefficient is calculated using a kinship window across the genome, and then “peak calling” algorithms can be used to identify regions where the estimated kinship is continuously at, around, or above 0.25. A sub-genome kinship coefficient is then determined for each kinship window. A kinship window can, in some embodiments, be based a given size, such as, for instance, a certain number of SNPs, or a certain distance, e.g., in centimorgan (cM), or a certain number of base pairs. In some embodiments, the sum of the width of the peaks in cM is then the estimated amount of shared DNA between the pair of individuals, which can then be translated into a kinship coefficient by, e.g., dividing the total amount of shared DNA, such as determined by peak calling algorithms, divided by 4.0, and then further divided by the total length of the genome inherited from one parent (in cM). Determining a kinship window value involves estimating the degree of relatedness between two individuals due to allele sharing above what one would expect by random chance.

In some embodiments, the kinship window is determined based on a number of SNPs. In some embodiments, the kinship window comprises at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 SNPs.

In some embodiments, the kinship window comprises between 5 and 500 SNPs, 5 and 450 SNPs, 5 and 400 SNPs, 5 and 350 SNPs, 5 and 300 SNPs, 5 and 250 SNPs, 5 and 200 SNPs, 5 and 175 SNPs, 5 and 150 SNPs, 5 and 125 SNPs, 5 and 100 SNPs, 10 and 500 SNPs, 10 and 450 SNPs, 10 and 400 SNPs, 10 and 350 SNPs, 10 and 300 SNPs, 10 and 250 SNPs, 10 and 200 SNPs, 10 and 175 SNPs, 10 and 150 SNPs, 10 and 125 SNPs, 10 and 100 SNPs, 25 and 500 SNPs, 25 and 450 SNPs, 25 and 400 SNPs, 25 and 350 SNPs, 25 and 300 SNPs, 25 and 250 SNPs, 25 and 200 SNPs, 25 and 175 SNPs, 25 and 150 SNPs, 25 and 125 SNPs, 25 and 100 SNPs, 50 and 500 SNPs, 50 and 450 SNPs, 50 and 400 SNPs, 50 and 350 SNPs, 50 and 300 SNPs, 50 and 250 SNPs, 50 and 200 SNPs, 50 and 175 SNPs, 50 and 150 SNPs, 50 and 125 SNPs, 50 and 100 SNPs, 75 and 500 SNPs, 75 and 450 SNPs, 75 and 400 SNPs, 75 and 350 SNPs, 75 and 300 SNPs, 75 and 250 SNPs, 75 and 200 SNPs, 75 and 175 SNPs, 75 and 150 SNPs, 75 and 125 SNPs, or 75 and 100 SNPs. In some embodiments, the kinship window comprises between 60 and 140 SNPs, 65 and 135 SNPs, 70 and 130 SNPs, 75 and 125 SNPs, 80 and 120 SNPs, 85 and 115 SNPs, 90 and 110 SNPs, or 95 and 105 SNPs.

In some embodiments, the kinship window comprises about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 270, about 275, about 280, about 285, about 290, about 295, or about 300 SNPs.

In some embodiments, the kinship window comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 SNPs. In some embodiments, the kinship window comprises 100 SNPs. In some embodiments, the kinship window comprises about 60 SNPs or about 100 SNPs.

In some embodiments, the kinship window comprises a length of at least 1 cM, at least cM, at least 10 cM, at least 15 cM, at least 20 cM, at least 25 cM, at least 30 cM, at least 35 cM, at least 40 cM, at least 45 cM, at least 50 cM, at least 55 cM, at least 60 cM, or at least 70 cM.

In some embodiments, the kinship window comprises a length of between 1 and 70 cM, 1 and 65 cM, 1 and 60 cM, 1 and 55 cM, 1 and 50 cM, 1 and 45 cM, 1 and 40 cM, 1 and 35 cM, 1 and 30 cM, 1 and 25 cM, 1 and 20 cM, 1 and 15 cM, 1 and 10 cM, 5 and 70 cM, 5 and 65 cM, 5 and 60 cM, 5 and 55 cM, 5 and 50 cM, 5 and 45 cM, 5 and 40 cM, 5 and 35 cM, 5 and 30 cM, 5 and 25 cM, 5 and 20 cM, 5 and 15 cM, 5 and 10 cM, 10 and 70 cM, 10 and 65 cM, 10 and 60 cM, 10 and 55 cM, 10 and 50 cM, 10 and 45 cM, 10 and 40 cM, 10 and 35 cM, 10 and 30 cM, and 25 cM, 10 and 20 cM, 10 and 15 cM, 15 and 70 cM, 15 and 65 cM, 15 and 60 cM, 15 and 55 cM, 15 and 50 cM, 15 and 45 cM, 15 and 40 cM, 15 and 35 cM, 15 and 30 cM, 15 and 25 cM, and 20 cM, 20 and 70 cM, 20 and 65 cM, 20 and 60 cM, 20 and 55 cM, 20 and 50 cM, 20 and 45 cM, 20 and 40 cM, 20 and 35 cM, 20 and 30 cM, or 20 and 25 cM.

In some embodiments, the kinship window comprises a length of about 1, about 5, about 10, about 15, about 20, about 25, about 30, about 35, about 40, about 45, about 50, about 55, about 60, about 65, or about 70 cM.

In some embodiments, the kinship window comprises a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 cM. In some embodiments, the kinship window comprises a length of 30 cM. In some embodiments, the kinship window comprises a length of about 30 cM.

In some embodiments, the kinship window comprises at least 1 million, 5 million, 15 million, 20 million, 25 million, 30 million, 35 million, 40 million, 45 million, 50 million, 55 million, 60 million, 65 million, or 70 million base pairs.

In some embodiments, the kinship window comprises between 1 and 70 million, 5 and 70 million, 10 and 70 million, 15 and 70 million, 20 and 70 million, 25 and 70 million, 30 and 70 million, 1 and 60 million, 5 and 60 million, 10 and 60 million, 10 and 55 million, 10 and 50 million, 10 and 45 million, 10 and 40 million, 10 and 35 million, 10 and 30 million, 15 and 70 million, 15 and 65 million, 15 and 60 million, 15 and 55 million, 15 and 50 million, 15 and 45 million, 15 and 40 million, 15 and 35 million, 15 and 30 million, 20 and 70 million, 20 and 65 million, 20 and 60 million, 20 and 55 million, 20 and 50 million, 20 and 45 million, 20 and 40 million, 20 and 35 million, 20 and 30 million, 25 and 70 million, 25 and 65 million, 25 and 60 million, 25 and 55 million, 25 and 50 million, 25 and 45 million, 25 and 40 million, 25 and 35 million, or 25 and 30 million base pairs.

In some embodiments, the kinship window comprises about 1 million, about 5 million, about 10 million, about 15 million, about 20 million, about 25 million, about 30 million, about 35 million, about 40 million, about 45 million, about 50 million, about 55 million, about 60 million, about 65 million, or about 70 million base pairs.

In some embodiments, the kinship window comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 million base pairs. In some embodiments, the kinship window comprises 30 million base pairs. In some embodiments, the kinship window comprises about 30 million base pairs.

In some embodiments, each of the plurality of kinship windows comprise a different set of SNPs from among the plurality of SNPs. In some embodiments, each of the plurality of kinship windows comprise a set of SNPs that comprises one or more SNPs that are shared with one or more other kinship windows from among the plurality of kinship windows. For instance, in some embodiments where each of the plurality of kinship windows comprise 100 SNPs, a first kinship window may comprise SNPs #1-100, a second kinship window may comprise SNPs #2-101, a third kinship window may comprise SNPs #3-102, and so on, such that each kinship window from among the plurality of kinship windows at least partially overlaps with one or more other kinship windows with regards to the SNPs they include. Accordingly, in some embodiments, each of the plurality of kinship windows overlaps with at least one other kinship window from among the plurality of kinship windows.

In some embodiments, each of the plurality of kinship windows overlaps with at least 5, at least 10, at least 20, at least 30, at least 40, at least 50, at least 60, at least 70, at least 80, at least 90, at least 100, or at least 100 other kinship window from among the plurality of kinship windows. In some embodiments, each of the plurality of kinship windows overlaps with N−1 other kinship window from among the plurality of kinship windows, wherein N is the number of SNPs contained within each of the plurality of kinship windows. In some embodiments, each of the plurality of kinship windows overlaps with a number of other kinship windows from among the plurality of kinship windows that is equal to the number of SNPs within each kinship window subtracted by 1.

In some embodiments, kinship windows on the ends of chromosomes may overlap with a smaller number of other kinship windows from among the plurality of kinship windows. Accordingly, in some embodiments, at least 30%, 40%, 50%, 60%, 70% 80%, 90%, 91%, 92%, 93%, 94%, 95%, 96%, 97%, 98%, or 99% of the kinship windows from among the plurality of kinship windows overlaps with N−1 other kinship window from among the plurality of kinship windows, wherein N is the number of SNPs contained within each of the plurality of kinship windows. In some embodiments, at least 30%, 40%, 50%, 60%, 70% 80%, 90%, 91%, 92%, 93%, 94%, 95%, 96%, 97%, 98%, or 99% of the kinship windows from among the plurality of kinship windows overlaps with a number of other kinship windows from among the plurality of kinship windows that is equal to the number of SNPs within each kinship window subtracted by 1.

In some embodiments, each of the plurality of kinship windows corresponds to a continuous segment of a chromosome. For instance, in some embodiments, each of the plurality of kinship windows will include the SNPs that are contained within a continuous (uninterrupted) segment of a chromosome. In other words, in these embodiments, a kinship window does not include SNPs from multiple different segments of multiple different chromosomes. In some embodiments, each of the plurality of kinship windows comprises SNPs that correspond to a continuous segment of a chromosome.

In some embodiments, the determining the kinship window value for each of the plurality of kinship windows is performed using algorithms and processes of, associated with, or derived from, PC-Relate. In some embodiments, the determining the kinship window value for each of the plurality of kinship windows is performed in accordance with algorithms and/or processes from PC-Relate. In some embodiments, the kinship window value represents the average value for the SNPs, i.e., the SNP values, within the kinship window, wherein the value for each SNP is 0 if the SNP is not shared with either alleles of the reference DNA profile, 0.25 if the SNP is shared with one allele of the reference DNA profile, or is 0.5 if the SNP is shared with both alleles of the reference DNA profile.

In some embodiments, the calculating the degree of relationship of the DNA profile to the reference DNA profile further comprises identifying one or more peaks across the plurality of kinship windows that exceed a kinship peak threshold value. In some embodiments, the calculating further comprises identifying a group of identified peaks comprising one or more identified peaks across the plurality of kinship windows that exceed a kinship peak threshold value. In some embodiments, the kinship peak threshold value is a value in the range of from about 0.15 to 0.25, such 0.15, 0.16, 0.17, 0.18, 0.19, 0.20, 0.21, 0.22, 0.23, 0.24, or 0.25. In some embodiments, the kinship peak threshold value is a value in the range of from about 0.20 to 0.25, such as 0.20, 0.205, 0.21, 0.215, 0.22, 0.225 0.23, 0.235, 0.24, 0.245, or 0.25. In some embodiments, the kinship peak threshold value is a value in the range of from about 0.21 to 0.25, such as 0.21, 0.215, 0.22, 0.225 0.23, 0.235, 0.24, 0.245, or 0.25.

In some embodiments, each of the identified peaks comprises a width in centimorgan (cM). In some embodiments, the width for each of the identified peaks is at least the width of a kinship window in cM. In some embodiments, each of the identified peaks has a width of at least 5, 10, 15, 20, 25, 35, 40, 45, 50, 55, 60, or 65 cM. In some embodiments, each of the identified peaks has a width of at least 20 cM. In some embodiments, at least one of the identified peaks has a width of at least 20, 21, 22, 23, 24, 25, 26, 27, 28, 29, 30, 31, 32, 33, 34, 35, 36, 37, 38, 39, 40, 41, 42, 43, 44, or 45 cM. In some embodiments, at least one of the identified peaks has a width of at least 25, 30, or 35 cM.

In some embodiments, each of the identified peaks has a minimum peak width. In some embodiments, the minimum peak width is, is about, is at least, or is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 cM. In some embodiments, the minimum peak width is or is about 20 cM. In some embodiments, the minimum peak width is or is about 15, 16, 17, 18, 19, or 20 cM.

In some embodiments, at least one of the identified peaks has a peak width of at least 20, 21, 22, 23, 24, 25, 26, 27, 28, 29, 30, 31, 32, 33, 34, 35, 36, 37, 38, 39, 40, 41, 42, 43, 44, or 45 cM. In some embodiments, at least one of the identified peaks has a peak width of at least 25, 26, 27, 28, 29, 30, 31, 32, 33, 34, or 35 cM. In some embodiments, at least one of the identified peaks has a peak width of at least 25 cM. In some embodiments, at least one of the identified peaks has a peak width of at least 30 cM. In some embodiments, at least one of the identified peaks has a peak width of at least 35 cM.

In some embodiments, the method further comprises determining whether one or more of the identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile. In some embodiments, the SNP threshold value is at least 0.90, 0.91, 0.92, 0.93, 0.94, 0.95, 0.96, 0.97, 0.98, or 0.99. In some embodiments, each of the identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile. In some embodiments, each of the identified peaks in the group of identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile.

In some embodiments, the method further comprises a step of excluding initially identified peaks from the group of identified peaks. In some embodiments, the excluding comprises excluding from the group of identified peaks any identified peaks that have a width below a minimum peak width. In some embodiments, the minimum peak width is or is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 cM. In some embodiments, the excluding comprises excluding from the group of identified peaks any identified peaks that have a shared SNP fraction value that does not exceed the SNP threshold value from the group of identified peaks.

In some embodiments, the width in cM of each of the identified peaks in the group of identified peaks is summed to determine the amount of shared DNA with one or more of the one or more reference DNA profile. In some embodiments, each of the identified peaks that are summed does not include any identified peak that has a width below a minimum peak width; and/or does not include any identified peak that has a shared SNP fraction value that does not exceed the SNP threshold value.

In some embodiments, the calculating comprises determining an overall kinship coefficient, wherein determining the overall kinship coefficient comprises calculating an overall kinship coefficient using the following formula: overall kinship coefficient=[the amount of shared DNA]/4.0/[total amount of genomic DNA]. In some embodiments, the total amount of genomic DNA is the total amount of genomic DNA that was inherited from one parent. In some embodiments, the total amount of genomic DNA is the total amount of genomic DNA that is expected to have been inherited from one parent. In some embodiments, the total amount of genomic DNA that is expected to have been inherited from one parent is or is about 3,560 cM.

### VI. Family Trees

In some embodiments, the methods provided herein further comprise generating a family tree comprising the DNA profile in relation to a reference DNA profile. In some embodiments, the methods provided herein further comprise generating a family tree comprising the DNA profile in relation to multiple reference DNA profiles. In some embodiments, the family tree comprises the DNA profile in relation to a reference DNA profile, wherein the reference DNA profile is a relative of the first degree, second degree, third degree, fourth degree, fifth degree, sixth degree, or seventh degree in relation to the DNA profile. In some embodiments, the family tree comprises the DNA profile in relation to multiple different reference DNA profiles, wherein each reference DNA profile is a relative of the first degree, second degree, third degree, fourth degree, fifth degree, sixth degree, or seventh degree in relation to the DNA profile.

### VII. Kits

Provided herein are kits comprising any of the primers, reagents or compositions described herein, which may further comprise instruction(s) on methods of using the kit, such as uses described herein. The kits described herein may also include other materials desirable from a commercial and user standpoint, including other buffers, diluents, filters, and package inserts with instructions for performing any methods described herein.

### VIII. Exemplary Embodiments

Among the provided embodiments are:
1. A method for performing DNA-based kinship analysis, comprising:
   providing a nucleic acid sample;
   amplifying the nucleic acid sample with a plurality of primers that specifically hybridize to a plurality of target sequences collectively comprising a plurality of single nucleotide polymorphisms (SNPs), thereby generating amplification products;
   sequencing the amplification products;
   determining the genotypes of the plurality of SNPs, thereby generating a DNA profile; and
   calculating the degree of relationship of the DNA profile to a reference DNA profile, wherein the calculating comprises determining a kinship window value for each of a plurality of kinship windows.
2. The method of embodiment 1, wherein the degree of relationship is represented by an overall kinship coefficient for the DNA profile with a reference DNA profile.
3. The method of embodiment 1 or embodiment 2, wherein each of the plurality of kinship windows comprise a different set of SNPs from among the plurality of SNPs.
4. The method of any one of embodiments 1-3, wherein each of the plurality of kinship windows corresponds to a continuous segment of a chromosome.
5. The method of any one of embodiments 1-4, wherein each of the plurality of kinship windows comprises SNPs that correspond to a continuous segment of a chromosome.
6. The method of any one of embodiments 1-5, wherein each of the plurality of kinship windows overlaps with at least one other kinship window from among the plurality of kinship windows.
7. The method of any one of embodiments 1-4, wherein the determining the kinship window value for each of the plurality of kinship windows is performed using algorithms and processes of, associated with, or derived from, PC-Relate.
8. The method of any one of embodiments 1-7, wherein the calculating further comprises identifying a group of identified peaks comprising one or more identified peaks across the plurality of kinship windows that exceed a kinship peak threshold value.
9. The method of embodiment 8, wherein the kinship peak threshold value is a value within the range of 0.15 to 0.25.
10. The method of embodiment 6 or embodiment 7, wherein each of the one or more identified peaks comprises a width in centimorgan (cM), and the width for each of the identified peaks is at least the width of a kinship window in cM.
11. The method of any one of embodiments 8-10, wherein each of the identified peaks has a width of at least 5, 10, 15, 20, 25, 35, 40, 45, 50, 55, 60, or 65 cM.
12. The method of any one of embodiments 8-11, wherein at least one of the identified peaks has a width of at least 20, 21, 22, 23, 24, 25, 26, 27, 28, 29, 30, 31, 32, 33, 34, 35, 36, 37, 38, 39, 40, 41, 42, 43, 44, or 45 cM.
13. The method of any one of embodiments 8-12, further comprising excluding from the group of identified peaks any identified peaks that have a width below a minimum peak width.
14. The method of embodiment 10, wherein the minimum peak width is or is about 5, 6, 7, 8, 9, 10, 11, 12, 13, 14, 15, 16, 17, 18, 19, 20, 21, 22, 23, 24, or 25 cM.
15. The method of any one of embodiments 8-14, further comprising determining whether one or more of the identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile.
16. The method of embodiment 15, wherein the SNP threshold value is at least 0.90, 0.91, 0.92, 0.93, 0.94, 0.95, 0.96, 0.97, 0.98, or 0.99.
17. The method of any one of embodiments 8-14, wherein each of the identified peaks has a shared SNP fraction value that exceeds a SNP threshold value, wherein the shared SNP fraction value is the fraction of SNPs within the identified peak out of the total number of SNPs within the identified peak that have at least one allele in common with a reference DNA profile.
18. The method of embodiment 17, wherein the SNP threshold value is at least 0.90, 0.91, 0.92, 0.93, 0.94, 0.95, 0.96, 0.97, 0.98, or 0.99.
19. The method of embodiment 15 or embodiment 16, further comprising excluding from the group of identified peaks any identified peaks that have a shared SNP fraction value that does not exceed the SNP threshold value from the group of identified peaks.
20. The method of any one of embodiments 8-19, wherein the width in cM of each of the identified peaks in the group of identified peaks is summed to determine the amount of shared DNA with a reference DNA profile.
21. The method of embodiment 20, wherein each of the identified peaks that are summed does not include any identified peak that has a width below a minimum peak width; and/or does not include any identified peak that has a shared SNP fraction value that does not exceed the SNP threshold value.
22. The method of embodiment 21, wherein the minimum peak width is or is about 5, 6, 7, 8, 9, 10, 11, 12, 13, 14, 15, 16, 17, 18, 19, 20, 21, 22, 23, 24, or 25 cM.
23. The method of embodiment 21, wherein the SNP threshold value is at least 0.90, 0.91, 0.92, 0.93, 0.94, 0.95, 0.96, 0.97, 0.98, or 0.99.
24. The method of any one of embodiments 1-23, wherein the calculating comprises determining an overall kinship coefficient, wherein determining the overall kinship coefficient comprises calculating an overall kinship coefficient using the following formula: overall kinship coefficient=[the amount of shared DNA]/4.0/[total amount of genomic DNA].
25. The method of embodiment 24, wherein the total amount of genomic DNA is the total amount of genomic DNA that was inherited from one parent.
26. The method of embodiment 24 or embodiment 25, wherein the total amount of genomic DNA is about 3,560 cM.
27. The method of any one of embodiments 1-26, wherein each of the plurality of kinship windows comprises between 25 and 200 SNPs.
28. The method of any one of embodiments 1-27, wherein each of the plurality of kinship windows comprises between 75 and 125 SNPs.
29. The method of any one of embodiments 1-28, wherein each of the plurality of kinship windows comprises about 60 SNPs or 100 SNPs.
30. The method of any one of embodiments 1-27, wherein each of the plurality of kinship windows comprises a length of between 5 and 70 centimorgan (cM).
31. The method of any one of embodiments 1-27 and 30, wherein each of the plurality of kinship windows comprises a length of between 20 and 40 cM.
32. The method of any one of embodiments 1-27, 30, and 31, wherein each of the plurality of kinship windows comprises a length of about 20 cM.
33. The method of any one of embodiments 1-27 and 30, wherein each of the plurality of kinship windows comprises between 5 and 70 million base pairs.
34. The method of any one of embodiments 1-27, 30, and 33, wherein each of the plurality of kinship windows comprises between 20 and 40 million base pairs.
35. The method of any one of embodiments 1-27, 30, 33, and 34, wherein each of the plurality of kinship windows comprises about 20 million base pairs.
36. A method for performing DNA-based kinship analysis, comprising:
   providing a nucleic acid sample;
   amplifying the nucleic acid sample with a plurality of primers that specifically hybridize to a plurality of target sequences collectively comprising a plurality of single nucleotide polymorphisms (SNPs), thereby generating amplification products;
   sequencing the amplification products;
   determining the genotypes of the plurality of SNPs, thereby generating a DNA profile; and
   calculating the degree of relationship of the DNA profile to a reference DNA profile, wherein the calculating comprises determining a chromosome-specific kinship value for each of two or more pairs of chromosomes.
37. The method of embodiment 36, wherein the two or more pairs of chromosomes comprises 10 or more, 11 or more, 12 or more, 13 or more, 14 or more, 15 or more, 16 or more, 17 or more, 18 or more, 19 or more, 20 or more, 21 or more, 22 or more, or 23 pairs of chromosomes.
38. The method of embodiment 36 or 37, wherein the two or more pairs of chromosomes comprises 22 pairs of chromosomes.
39. The method of any one of embodiments 36-38, wherein the calculating comprises determining a total number of shared SNPs between the DNA profile and the reference DNA profile.
40. The method of any one of embodiments 36-39, wherein the determining the chromosome specific kinship value is based on a comparison of the shared SNPs between the DNA profile and the reference DNA profile.
41. The method of embodiment 40, wherein the comparison comprises determining a total number of overlapping SNPs between the DNA profile and the reference DNA profile.
42. The method of any one of embodiments 36-41, wherein the determining the chromosome specific kinship value for each of the two or more pairs of chromosomes is performed using algorithms and processes of, associated with, or derived from, PC-Relate.
43. The method of any one of embodiments 36-42, wherein the calculating an overall CSKP score for the DNA profile in comparison to the reference DNA profile comprises the use of a random forest model and a chromosome specific kinship value for each of the two or more pairs of chromosomes.
44. The method of embodiment 43, wherein the generating a CSKP model comprises:
   (a) calculating a mean and standard deviation of chromosome kinship for each chromosome contained within an unrelated sample training set, wherein the unrelated sample training set comprises samples from unrelated individuals;
   (b) calculating a z-score for each chromosome kinship;
   (c) calculating a log survival function on the z-score;
   (d) calculating a sum of the log survival function for each of the two or more chromosomes;
   (e) performing a logistic regression on the sum of the log survival function; and
   (f) training a random forest model on overall kinship, log probability from logistic regression analysis, and total overlapping SNPs between samples.
45. The method of embodiment 44, wherein the calculating an overall CSKP score comprises:
   (a) determining a chromosome specific kinship value for each of the two or more pairs of chromosomes and calculating a z-score for each chromosome specific kinship value based on the mean and standard deviation of chromosome kinship for the unrelated sample training set;
   (b) calculating a log survival function value for the z-score of each chromosome specific kinship value and summing the log survival function values;
   (c) calculating a log probability using the summed value of log survival function values; and
   (d) determining an overall CSKP score using the random forest model based on the log probability, the total number of shared SNPs between the DNA profile and the reference DNA profile, and the overall kinship value.
46. The method of any one of embodiments 36-42, wherein the calculating an overall CSKP score for the DNA profile in comparison to the reference DNA profile comprises the use of a random forest model and a chromosome specific kinship value for each of the two or more pairs of chromosomes.
47. The method of embodiment 46, wherein the calculating an overall CSKP score comprises:
   (a) determining a chromosome specific kinship value for each of the two or more pairs of chromosomes and calculating a z-score for each chromosome specific kinship value based on a mean and standard deviation of chromosome kinship for an unrelated sample training set, wherein the mean and standard deviation of chromosome kinship for the unrelated sample training set were determined by a CSKP model comprising the steps of:
      (i) calculating a mean and standard deviation of chromosome kinship for each chromosome contained within an unrelated sample training set, wherein the unrelated sample training set comprises samples from unrelated individuals;
      (ii) calculating a z-score for each chromosome kinship;
      (iii) calculating a log survival function on the z-score;
      (iv) calculating a sum of the log survival function for each of the two or more chromosomes;
      (v) performing a logistic regression on the sum of the log survival function; and
      (vi) training a random forest model on overall kinship, log probability from logistic regression analysis, and total overlapping SNPs between samples; and
   (b) calculating a log survival function value for the z-score of each chromosome specific kinship value and summing the log survival function values;
   (c) calculating a log probability using the summed value of log survival function values; and
   (d) determining the overall CSKP score using the random forest model based on the log probability, the total number of shared SNPs between the DNA profile and the reference DNA profile, and the overall kinship value.
48. The method of any one of embodiments 36-47, wherein the overall CSKP score for the DNA profile each reference DNA profile represents the relatedness of the DNA profile with the reference DNA profile.
49. The method of any one of embodiments 1-48, wherein the plurality of SNPs comprises between 5,000 and 50,000 SNPs.
50. The method of any one of embodiments 1-49, wherein the plurality of SNPs comprises between 5,000 and 15,000 SNPs.
51. The method of any one of embodiments 1-50, wherein the plurality of SNPs comprises between 9,000 and 11,000 SNPs.
52. The method of any one of embodiments 1-51, wherein the amplification is carried out in one or more multiplex PCR reactions
53. The method of any one of embodiments 1-52, wherein the sequencing is conducted using massively parallel sequencing (MPS).
54. The method of any one of embodiments 1-53, wherein the sequencing does not comprise whole genome sequencing (WGS).
55. The method of any one of embodiments 1-54, wherein the nucleic acid sample comprises genomic DNA.
56. The method of any one of embodiments 1-55, wherein the nucleic acid sample comprises one or more enzyme inhibitors.
57. The method of embodiment 56, wherein the one or more enzyme inhibitors comprise one or more inhibitors selected from the group consisting of hematin, humic acid, indigo, and tannic acid.
58. The method of any one of embodiments 1-57, wherein the nucleic acid sample comprises low-quality nucleic acid molecules and/or low quantity nucleic acid molecules.
59. The method of embodiment 58, wherein the low quality nucleic acid molecules are degraded genomic DNA and/or fragmented genomic DNA.
60. The method of any one of embodiments 1-59, wherein the nucleic acid sample is a forensic sample.
61. The method of any one of embodiments 1-60, wherein the nucleic acid sample is derived from a buccal swab, paper, fabric, or other substrate that is impregnated with saliva, blood, or other bodily fluid.
62. The method of any one of embodiments 1-61, wherein the nucleic acid sample comprises between or between about 50 pg and 100 ng of genomic DNA.
63. The method of any one of embodiments 1-62, wherein the nucleic acid sample comprises between or between about 100 pg and 5 ng of genomic DNA.
64. The method of any one of embodiments 1-63, wherein the nucleic acid sample comprises at or about 1 ng of genomic DNA.
65. The method of any one of embodiments 1-64, wherein the plurality of SNPs comprises kinship SNPs.
66. The method of any one of embodiments 1-65, wherein the plurality of SNPs comprises kinship SNPs, ancestry SNPs, identity SNPs, phenotype SNPs, X-SNPs, and Y-SNPs.
67. The method of any one of embodiments 1-66, wherein the plurality of SNPs comprises SNPs selected from one or more of the groups consisting of kinship SNPs, ancestry SNPs, identity SNPs, phenotype SNPs, X-SNPs, and Y-SNPs.
68. The method of any one of embodiments 1-67, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.
69. The method of any one of embodiments 1-68, wherein the DNA profile in relation to the reference DNA profile is a relative of the first degree, second degree, third degree, fourth degree, fifth degree, sixth degree, or seventh degree.
70. The method of embodiment 69, wherein the DNA profile in relation to the reference DNA profile is a relative of the fourth degree or fifth degree.
71. The method of any one of embodiments 1-70, further comprising generating a family tree comprising the DNA profile in relation to the reference DNA profile.

### IX. 1. EXAMPLES

The following examples are included for illustrative purposes only and are not intended to limit the scope of the invention.

### EXAMPLE 1 Generation of Sequence Libraries and Determination of Sensitivity

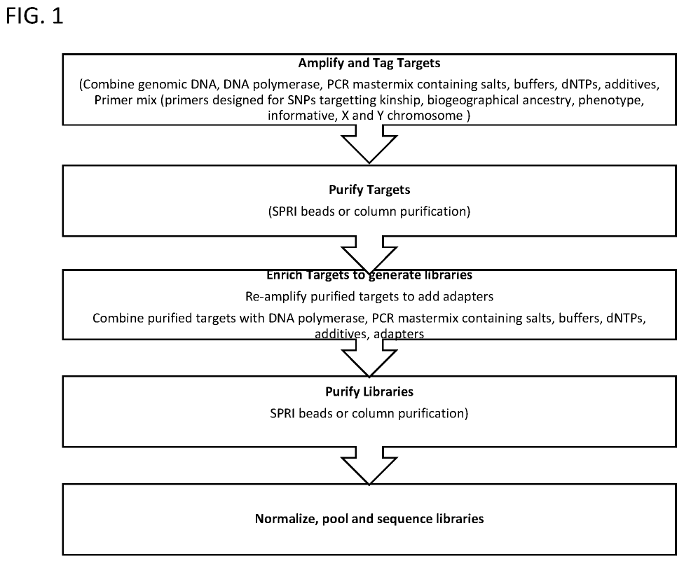

This Example describes a method of determining the sensitivity of the multiplex polymerase chain reaction described herein to generate libraries capable of being sequenced. FIG. 1 depicts an exemplary schematic of the method for generating a library capable of being sequenced described in this Example.

### A. PCR Amplification of Genomic DNA Target

A multiplex polymerase chain reaction was performed to amplify 10,230 individual amplicons in a genomic DNA sample. Each primer pair was designed to selectively hybridize to, and promote amplification of a specific single nucleotide polymorphism (SNP) of the genomic DNA sample. A range of input genomic DNA was tested from 50 ng to 50 pg, more specifically, 5 ng, 2.5 ng, 1 ng, 500 pg, 250 pg, 100 pg and 50 pg). Briefly, 18.5 ml of a PCR mastermix containing sufficient buffer, dNTPs, MgCl2, salts and PCR additives such as glycerol was added to a single well of a 96-well PCR plate. 5 microliters of Primer Pool, containing 10,530 primer pairs, 2-4Units of a DNA polymerase such as Phusion hot start DNA polymerase (Thermo Fisher, cat #F549L or any other thermostable DNA polymerase, 50 ng to 50 pg genomic DNA were also added.

The PCR plate was sealed and loaded into a thermal cycler (Veriti 96-well thermal cycler, Thermo Fisher Scientific, 4413964) and run on the temperate profile described below to generate the amplicon library.
98° C. for 3 minutes
18 cycles of:
96° C. for 45 seconds
80° C. for 10 seconds
54° C. for 4 minutes with applicable ramp mode
66° C. for 90 seconds with applicable ramp mode
68° C. for 10 minutes
Hold at 4° C.

After cycling, the amplicon library was held at 2-8° C. until proceeding to the purification step outlined below.

B. Purification of Amplicons from Input DNA and Primers

Two rounds of clean-up using MagBind Total Pure NGS beads (Omega Biotek, M1378-02) binding, wash, and elution at 1.6X and 0.6X volume ratios were found to remove genomic DNA and unbound or excess primers. The amplification and purification step outlined herein produces amplicons of about 150-350 bp in length. Purified amplicons are then used in a second round of PCR to add adapters for sequencing.

C. Enrichment of Purified Amplicons to Generate Libraries Capable of being Sequenced

A second round of PCR amplification is performed by combining 25 ml of purified amplicons from step above with 5 ml of adapters provided in Forenseq Kintelligence kit (Verogen PN: V16000120) and 20 ml of KPCR2 mastermix provided in Forenseq Kintelligence kit (Verogen PN: V16000120) in a 96 well PCR plate. The PCR plate was sealed and loaded into a thermal cycler (Veriti 96-well thermal cycler, Thermo Fisher Scientific, 4413964) and run on the temperate profile described below to generate the amplicon library.
98° C. for 30 seconds
15 cycles of:
98° C. for 20 seconds
66° C. for 30 seconds
72° C. for 30 seconds
72° C. for 1 minute
Hold at 4° C.

The libraries were purified using MagBind Total Pure NGS beads (Omega Biotek, M1378-02) binding, wash, and elution at 1X. The purified libraries were quantitated, normalized, denatured and diluted as per instructions given in Forenseq Kintelligence kit User Guide (Verogen PN: V16000120, the contents of which are hereby incorporated by reference in their entirety).

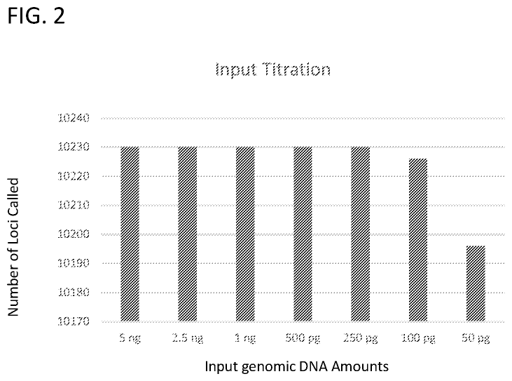

The denatured libraries were sequenced as per instructions on MiSeq FGx Sequencing System Reference Guide (document #VD2018006, the contents of which are hereby incorporated by reference in their entirety). As shown in FIG. 2, input genomic DNA quantities were similar across a range of input titrations.

Results were analyzed using the Forenseq Universal Analysis Software 2.1 (Verogen, San Diego,CA) following the instructions outlined in Forenseq Universal Analysis Software 2.1, and provided in Reference Guide Document #VD2019002, the contents of which are hereby incorporated by reference in their entirety.

### EXAMPLE 2 Generation of Sequence Libraries Using Degraded DNA

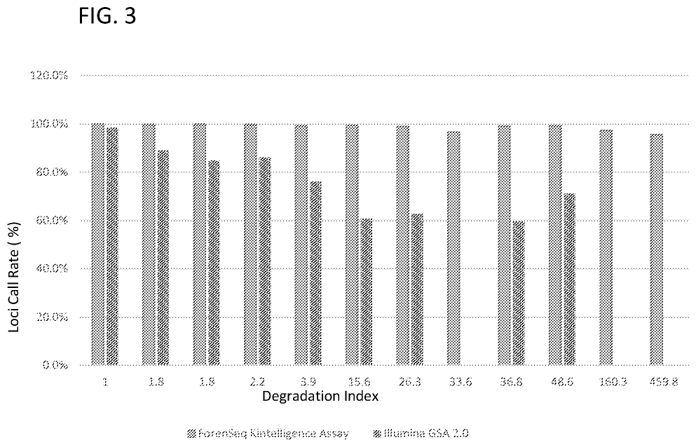

This Example describes the sequencing of DNA from low quantity and highly degraded samples. Degraded DNA A series of degraded blood DNA was obtained from Innogenomics (New Orleans, LA). The DNA samples were used to generate sequencing libraries as described in Example 1, with the exception that primer pairs for 10,327 loci were used in this example. The percentage of Loci detected (call rate) with degraded DNA using the assay described herein compared to Microarray (GSA) call rate is shown in FIG. 3. The degradation Index (DI) is shown on x-axis and the number of detected loci on Y-axis. These results show that even with highly degraded DNA with a DI of 158.3, the assay detected 9167 loci, which is sufficient to upload to the genealogy database to search for relatives. The alternative technologies such as Microarrays failed to detect any loci in samples with high degradation index.

### EXAMPLE 3 Assessment of Activity of Inhibitors on Library Preparation

This Example describes assessment of the effect of PCR inhibitors on the preparation of libraries disclosed herein. DNA samples from crime scenes often contain co-purified impurities which inhibit PCR. PCR inhibition is the most common cause of PCR failure when adequate copies of DNA are present. Humic compounds, a series of substances produced during decay process have been considered as the materials contaminating DNA in soil, natural waters and recent sediments. Other common inhibitors include hematin (from blood), indigo (from blue jeans) and tannic acid.

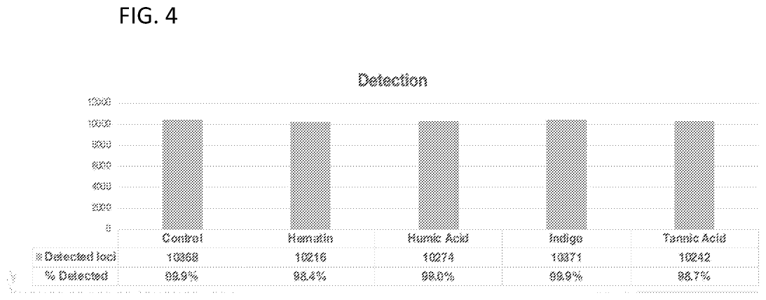

To assess the impact of inhibitors commonly found in forensic samples, library preparation was performed as described in Example 1, with the exceptions of 200 uM Hematin, 50 ng/uL Humic Acid, 133 uM Indigo, 16 uM Tannic Acid were spiked into the “Amplify and Tag targets” step above and primer pairs for 10380 loci were used. Results are shown in FIG. 4, with a PCR reaction without any inhibitors is labeled as Control.

### EXAMPLE 4 Determining Kinship Using Chromosome-Specific Kinship Probabilities (CSKP)

A method for determining overall kinship was developed that employs a scoring method called chromosome-specific kinship probabilities (CSKP). This approach determines overall kinship confidence by assessing kinship probabilities in a chromosome-by-chromosome manner (to generate a chromosome specific kinship value) and then using those individual values to calculate an overall CSKP confidence value (also referred to herein as an overall CSKP score), which can be used to filter kinship matches between the sample's DNA profile and one or more reference DNA profiles.

A CSKP model was built by performing the steps of: (1) calculating the mean and standard deviation of chromosome kinship for each chromosome using an unrelated sample training set, where each chromosome is from an unrelated sample within the unrelated sample training set, and where chromosome kinship is based on the number of shared SNPs; (2) calculating the z-score for each chromosome kinship; (3) calculating the log survival function on the z-score, where the log probabilities for a distribution of related individuals have a z-score greater than a distribution of unrelated individuals; (4) calculating the sum of the log survival function for all of the chromosomes, wherein the sum reflects the product of all probabilities that a specific chromosome kinship value is from the “unrelated” distribution; (5) performing a logistic regression on the sum; and (6) training a random forest on overall kinship, log probability from logistic regression analysis, and the total overlapping SNPs between samples, where overall kinship reflects the kinship value based on the sharing of all SNPs within the genome, and where total overlapping SNPs reflects how many total SNPs were shared between the two individuals throughout the entire genome. This CSKP model is then used for calculating the overall CSKP score when conducting kinship analyses between a DNA profile and one or more reference DNA profiles. The CSKP model only needs to be performed once and then kinship for subsequent samples of interest can be determined using this training model.

For each kinship calculation, the overall CSKP score for a DNA profile in comparison to a reference DNA profile is calculated by performing the steps of (1) Determining the individual chromosome specific kinship values for each chromosome and calculating the z-score based on the training mean and standard deviations for the unrelated set for the CSKP model previously generated; (2) Calculating the log survival function for the chromosome specific z-scores and summing the values; (3) Calculating the log probability using the summed z-scores in the previously described logistic regression model (the CSKP model); and (4) Taking the log probability, number of overlapping SNPs, and overall kinship, and running it through the random forest model to yield the overall CSKP score, where the overall kinship reflects the kinship value based on the sharing of all SNPs within the genome, and where total overlapping SNPs reflects how many total SNPs were shared between the two individuals throughout the entire genome.

Each of these steps, individually, were performed using calculations and algorithms that are well-known to those skilled in the art.

This CSKP scoring method was built with the goal of successfully filtering distant relatives of the fifth degree, but is expected to also work for distant relatives of the first degree, second degree, third degree, fourth degree, sixth degree, and greater degrees as well.

Using the CSKP method described above, an accuracy of 93% was achieved with an F1 of 0.68%. This was more accurate than a decision tree that was trained solely on overall kinship on a genome-wide basis, which achieved a lower accuracy of 77% with an F1 score of 0.77%.

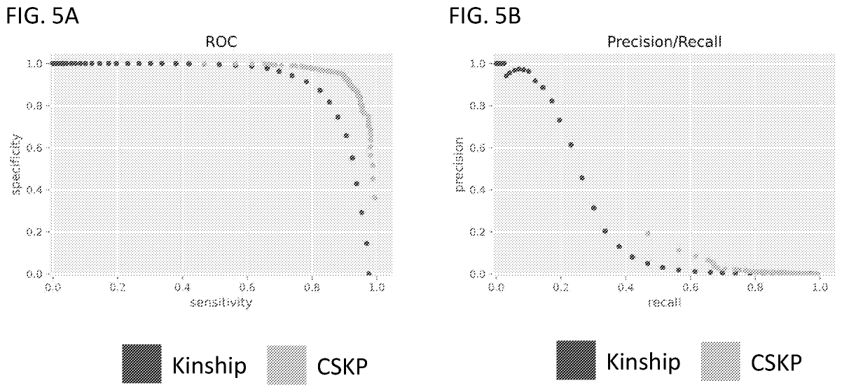

Using the same training set having known fifth degree true positive relatives and known unrelated individuals, the CSKP approach was compared with an approach based solely on overall kinship using two common prediction models. A receiver operating characteristic (ROC) curve for specificity vs sensitivity (FIG. 5A), and a precision-recall curve for precision vs recall (FIG. 5B) were generated.

A ROC curve is a plot showing the true positive rate (sensitivity) vs the true negative rate (specificity). In other words, the ROC curve provides a curve showing the probability that a sample will be positive when the individuals are truly related (sensitivity) vs the probability that a sample will be negative when the individuals are truly not related (specificity). Each of the points on the ROC curve reflects a pair of specificity and sensitivity values at various possible thresholds. As shown in FIG. 5A, the CSKP approach was shown to be superior to the overall kinship approach by maintaining higher specificity as the sensitivity increases, and vice versa, i.e., the area under the curve (AUC) is greater for the CSKP approach. Thus, the CSKP approach was shown to provide for improved specificity and sensitivity over the approach based on overall kinship alone (genome-wide approach).

A precision-recall curve is a plot having precision values (also called the positive predictive value) on the y-axis and recall values (also called sensitivity or the true positive rate) on the x-axis. A precision-recall curve is typically more useful than a ROC curve when there is a high number of true negatives in the sample population, which, for a ROC curve, could lead to a high specificity value that would still yield a high number of false positives. The precision values are calculated as follows: TP/(TP+FP); and the recall values are calculated as follows: TP/(TP+FN), where TP=true positive, FN=false negative, and FP=false positive. The precision value reflects how well the model is able to only classify truly positive samples, i.e., truly related individuals, as positive and not to incorrectly label negative samples as positive. The recall value reflects how well the model is able to identify all truly positive samples, i.e., truly related individuals. Each of the points on the precision-recall curve reflects a pair of precision and recall values at various possible thresholds. As shown in FIG. 5B, the CSKP approach was shown to be superior to the overall genome-wide kinship approach in its predictive value. For instance, at 45% recall, precision is significantly greater for the CSKP approach than the approach based on overall genome-wide kinship alone (FIG. 5B).

### EXAMPLE 5 Determining Kinship Using Sub-Genome Kinship Coefficients

A method for determining overall kinship was developed using sub-genome kinship coefficients. The approach using sub-genome kinship coefficients generates a series of kinship values based on a subset of SNPs from a total set of SNPs. Each subset of SNPs is located within each of a plurality of overlapping kinship windows throughout the genome, thereby covering the entire genome through a plurality of the kinship windows, with each kinship window providing a kinship window value. Each of the series of kinship window values is combined in order to give information about region-specific “hot spots” of sequence similarity, i.e., where there is shared DNA. This also allows for the determination of an overall kinship coefficient after assessing the kinship window values for each of the plurality of kinship windows. In contrast, a genome-wide kinship coefficient, as used in certain existing methods, is based on the SNPs across the genome as a whole, rather than in smaller windows of SNPs.

For each kinship window, a kinship window value is generated based on the number of shared SNPs within the kinship window. A kinship window of a given size is used. In some applications, kinship windows of 50-100 SNPs per kinship window are used. In other applications, kinship windows of 10-30 centimorgan (cM) per kinship window are used. In certain experiments, a kinship window containing 60 SNPs was used, with a different 60-SNP kinship window starting at every SNP beginning at one end of each chromosome, which resulted in almost as many kinship windows as the total number of SNPs assessed. This approach allows for generating multiple kinship window values that overlap each SNP, which allows for generating a moving average of kinship along each entire chromosome.

Each kinship window value is determined based on the shared SNPs within the window, such as by using available methods, including algorithms and processes of, associated with, or derived from, PC-Relate. Conceptually, for each SNP, a value of zero (0) is assigned if neither of the two chromosomes is shared between the two individuals at that SNP, a value of 0.25 is assigned if one of the two chromosomes is shared between the two individuals at that SNP, and a value of 0.5 is assigned if both of the two chromosomes is shared between the two individuals at that SNP. As such, a kinship window includes SNPs each having one of these SNP values, and calculations involving these SNP values can be used to calculate a kinship window value, which represents an estimate of the degree of relatedness of the DNA segment that contains the SNPs within the kinship window.

In some experiments, each kinship window value is determined based on the number of SNPs shared within the kinship window, optionally with SNP values associated with them. The kinship window value for each kinship window is calculated using the algorithms and processes of, associated with, or derived from, the PC-Relate method. See, e.g., Conomos et al., Model-free Estimation of Recent Genetic Relatedness, Am. J. Hum. Genet., 98 (1): 127-148 (2016).

Accordingly, a kinship window value is generated for each kinship window by taking into account the shared SNPs within the kinship window using available methods. Well-understood “peak calling” algorithms can then be used to identify regions (or peaks) in the genome, represented by overlapping kinship windows, where the estimated kinship, i.e., kinship window value, is continuously at, around, or above a certain threshold, e.g., 0.22, for that region in the genome. For instance, in some experiments, a peak is identified when a kinship window value exceeds a certain threshold, e.g., 0.22, and then the peak continues so long as the additional overlapping kinship window values also exceed the threshold, and then the peak ends when the kinship window values drop below the threshold for at least N consecutive kinship windows, where N is any suitable number, such as 10 in some experiments.

As an alternative to a “peak calling” algorithm, such as described above, another approach that could be used to identify peaks of shared DNA is Circular Binary Segmentation, which is a common algorithm used in Copy Number Calling to identify the boundaries of copy number changes that occur.

The identified peaks are then post-filtered using the expectation that in a DNA segment shared by inheritance the two samples will share at least one allele in common at each SNP. The total number of SNPs within the peak is calculated along with the number of those SNPs at which the pair of samples share at least one allele in common. If the fraction of SNPs with at least one shared allele in common relative to the total number of SNPs within the peak is below a threshold value (e.g., 0.9, 0.95, or 0.99), the peak is discarded. The sum of the width of the retained peaks in cM is then used as the estimated amount of shared DNA between the two individuals, and this is then translated into an overall kinship coefficient by a simple mathematical formula, e.g., overall kinship coefficient=shared cM/4.0/length of human genome in cM.

Using this sub-genome kinship coefficient method, two samples that are truly unrelated, i.e., are a true negative for kinship, should have values within a given kinship window that are 0. However, due to noise, imperfect modeling, and other sources of error, values within a kinship window, or across overlapping kinship windows, may reach values higher than 0, but should rarely peak near 0.25. In contrast, it is expected that samples from two truly related individuals who are distantly related, e.g., of the fourth, fifth, or sixth degree, would exhibit values within a kinship window, or across overlapping kinship windows, that mirror the pattern of kinship for truly unrelated individuals, i.e., have a kinship coefficient of 0 or close to zero, in regions of the genome where they do not share DNA by inheritance, and have a pattern of kinship for truly related individuals, e.g., have a kinship coefficient at, around, or above 0.25, within regions of the genome where shared DNA by inheritance is present. The sub-genome kinship coefficient approach can identify these regions where shared DNA by inheritance is present because it breaks up the kinship analysis into a series of overlapping kinship windows, i.e., segments, which is reflective of the segmented way in which shared DNA is present when comparing distantly related individuals, e.g., of the fourth, fifth, or sixth degree.

### EXAMPLE 6 Determining Kinship in an Exemplary Study Using the Sub-Genome Approach, the CSKP Approach, and the Existing Genome-Wide Kinship Approach

Kinship was determined in an exemplary study using each of the two approaches described herein, i.e., the CSKP approach and the sub-genome kinship coefficient approach, as well as using the existing genome-wide kinship approach for comparison. The approach referred to as the existing genome-wide kinship approach utilizes the algorithms and processes of, associated with, or derived from, the PC-Relate method.

A dataset comprised of several thousand samples was examined in this study, which contained 1,559 related sample pairs with genetic sharing in the range of 100-450 cM, and 11,531,764 unrelated sample pairs.

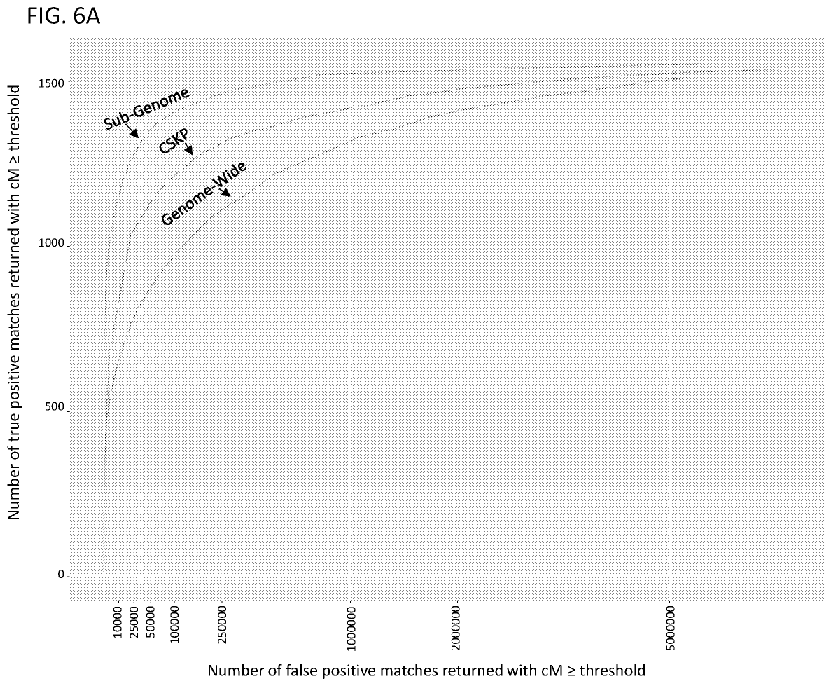

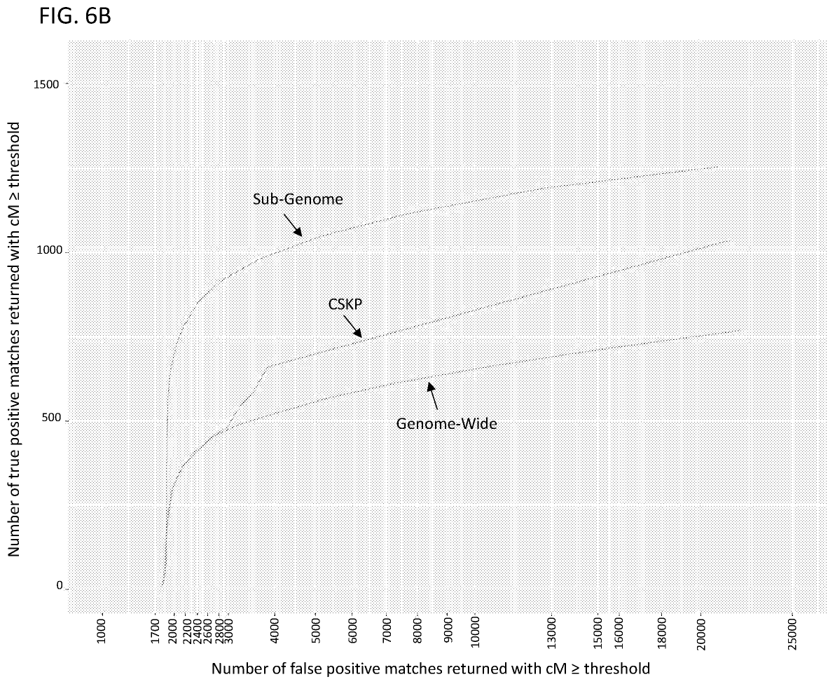

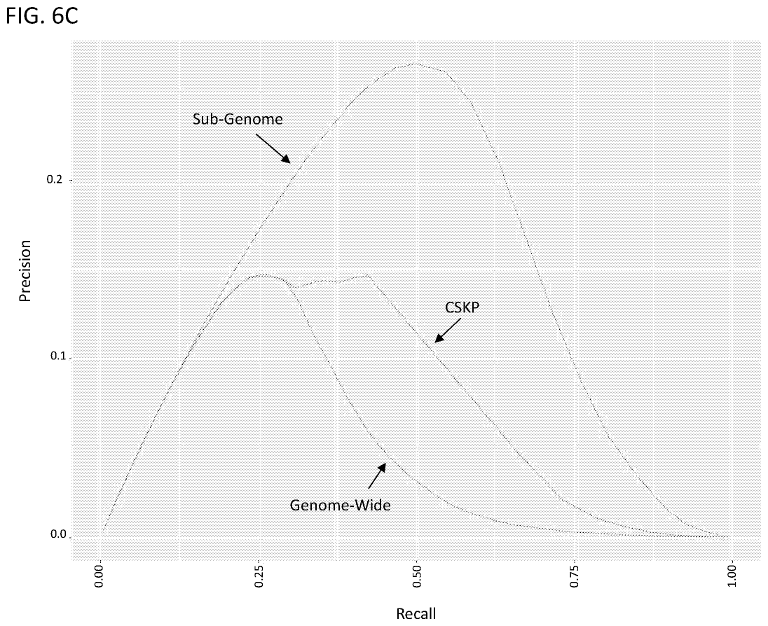

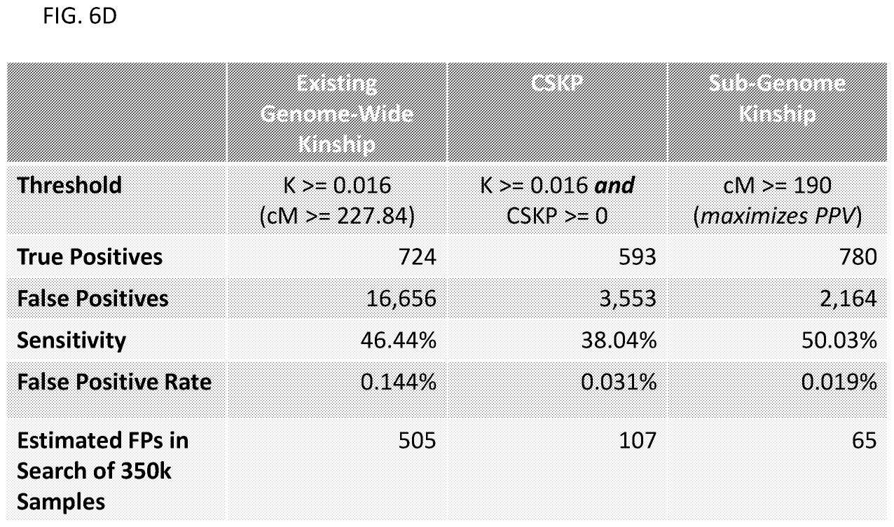

The sub-genome kinship coefficient method was used to estimate genetic sharing in cM between each pair of samples, i.e., to estimate an overall kinship coefficient. The resulting set of data from 1,559 related sample pairs and 11,531.764 unrelated sample pairs was filtered to estimated genetic sharing at certain thresholds, including ≥0 cM, ≥10 cM, ≥20 cM, etc., up to ≥500 cM, and calculated the sensitivity, specificity, and precision at each threshold. This data was used to generate ROC and precision-recall curves for the sub-genome kinship coefficient approach, which involves the use of kinship windows. The same experimental procedure was also performed using the CSKP approach, as well as using a genome-wide kinship approach, i.e., an existing, non-windowed approached, in order to generate matching ROC and precision-recall curves for all three approaches (FIGS. 6A-C). FIGS. 6A and 6B show the number of true positive matches returned with a cM≥the threshold on the y-axis, and the number of false positive matches returned with a cM≥the threshold on the x-axis. FIG. 6C shows precision on the y-axis, and recall on the x-axis. As shown in FIGS. 6A and 6B, the sub-genome and CSKP approaches were shown to be superior to the existing genome-wide approach using PC-Relate in their predictive value, as indicated by the ROC curves, e.g., by having larger areas under the curve. Moreover, as shown in FIG. 6C, the sub-genome and CSKP approaches were also shown to be superior to the existing genome-wide approach using PC-Relate in their predictive value, as indicated by the precision-recall curves. For instance, at 50% (0.50) recall, precision is substantially greater with the sub-genome and CSKP approaches than the genome-wide PC-Relate approach.

Finally, a filtering threshold was selected for the sub-genome approach that closely approximates the sensitivity of the existing genome-wide kinship approach and threshold (FIG. 6D). FIG. 6D presents the number of true positives, false positives, sensitivity, false positive rate, and the estimated number of how many false positives (FPs) each approach would produce (on average) when queried against a 350,000 sample database. As shown in FIG. 6D, the number of false positives is substantially less when using the CSKP approach (3,553) or the sub-genome approach (2,164) as compared to the existing genome-wide approach (16,656). Moreover, the estimated number of false positives in a search of 350,000 samples is substantially less when using the CSKP approach (107) or the sub-genome approach (65) as compared to the existing genome-wide approach (505) (FIG. 6D).

This study demonstrates that the CSKP and sub-genome approaches are superior to the existing genome-wide approach by greatly reducing false positive rates while maintaining sensitivity for identifying true positives.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for performing DNA-based kinship analysis, comprising:
providing a nucleic acid sample;
amplifying the nucleic acid sample with a plurality of primers that specifically hybridize to a plurality of target sequences collectively comprising a plurality of single nucleotide polymorphisms (SNPs), thereby generating amplification products;
sequencing the amplification products;
determining the genotypes of the plurality of SNPs, thereby generating a DNA profile; and
calculating the degree of relationship of the DNA profile to a reference DNA profile, wherein the calculating comprises determining a kinship window value for each of a plurality of kinship windows.

2. The method of claim 1, wherein the degree of relationship is represented by an overall kinship coefficient for the DNA profile with the reference DNA profile.

3. The method of claim 1 or claim 2, wherein each of the plurality of kinship windows comprise a different set of SNPs from among the plurality of SNPs.

4. The method of any one of claims 1-3, wherein each of the plurality of kinship windows corresponds to a continuous segment of a chromosome.

5. The method of any one of claims 1-4, wherein each of the plurality of kinship windows comprises SNPs that correspond to a continuous segment of a chromosome.

6. The method of any one of claims 1-5, wherein each of the plurality of kinship windows overlaps with at least one other kinship window from among the plurality of kinship windows.

7. The method of any one of claims 1-4, wherein the determining the kinship window value for each of the plurality of kinship windows is performed using algorithms and processes of, associated with, or derived from, PC-Relate.

8. The method of any one of claims 1-7, wherein the calculating further comprises identifying a group of identified peaks comprising one or more identified peaks across the plurality of kinship windows that exceed a kinship peak threshold value.

9. The method of claim 8, wherein the kinship peak threshold value is a value within the range of 0.15 to 0.25.

10. The method of claim 6 or claim 7, wherein each of the one or more identified peaks comprises a width in centimorgan (cM), and the width for each of the identified peaks is at least the width of a kinship window in cM.

11. The method of any one of claims 8-10, wherein each of the identified peaks has a width of at least 5, 10, 15, 20, 25, 35, 40, 45, 50, 55, 60, or 65 cM.

12. The method of any one of claims 8-11, wherein at least one of the identified peaks has a width of at least 20, 21, 22, 23, 24, 25, 26, 27, 28, 29, 30, 31, 32, 33, 34, 35, 36, 37, 38, 39, 40, 41, 42, 43, 44, or 45 cM.

13. The method of any one of claims 8-12, further comprising excluding from the group of identified peaks any identified peaks that have a width below a minimum peak width.

14. The method of claim 10, wherein the minimum peak width is or is about 5, 6, 7, 8, 9, 10, 11, 12, 13, 14, 15, 16, 17, 18, 19, 20, 21, 22, 23, 24, or 25 cM.

15. The method of any one of claims 8-14, further comprising determining whether one or more of the identified peaks has a shared SNP fraction value that exceeds a SNP threshold value, wherein the shared SNP fraction value is the fraction of SNPs within the identified peak out of the total number of SNPs within the identified peak that have at least one allele in common with the reference DNA profile.

16. The method of claim 15, wherein the SNP threshold value is at least 0.90, 0.91, 0.92, 0.93, 0.94, 0.95, 0.96, 0.97, 0.98, or 0.99.

17. The method of any one of claims 8-14, wherein each of the identified peaks has a shared SNP fraction value that exceeds a SNP threshold value, wherein the shared SNP fraction value is the fraction of SNPs within the identified peak out of the total number of SNPs within the identified peak that have at least one allele in common with the reference DNA profile.

18. The method of claim 17, wherein the SNP threshold value is at least 0.90, 0.91, 0.92, 0.93, 0.94, 0.95, 0.96, 0.97, 0.98, or 0.99.

19. The method of claim 15 or claim 16, further comprising excluding from the group of identified peaks any identified peaks that have a shared SNP fraction value that does not exceed the SNP threshold value from the group of identified peaks.

20. The method of any one of claims 8-19, wherein the width in cM of each of the identified peaks in the group of identified peaks is summed to determine the amount of shared DNA with the reference DNA profile.

21. The method of claim 20, wherein each of the identified peaks that are summed does not include any identified peak that has a width below a minimum peak width; and/or does not include any identified peak that has a shared SNP fraction value that does not exceed the SNP threshold value.

22. The method of claim 21, wherein the minimum peak width is or is about 5, 6, 7, 8, 9, 10, 11, 12, 13, 14, 15, 16, 17, 18, 19, 20, 21, 22, 23, 24, or 25 cM.

23. The method of claim 21, wherein the SNP threshold value is at least 0.90, 0.91, 0.92, 0.93, 0.94, 0.95, 0.96, 0.97, 0.98, or 0.99.

24. The method of any one of claims 1-23, wherein the calculating comprises determining an overall kinship coefficient, wherein determining the overall kinship coefficient comprises calculating an overall kinship coefficient using the following formula: overall kinship coefficient=[the amount of shared DNA]/4.0/[total amount of genomic DNA].

25. The method of claim 24, wherein the total amount of genomic DNA is the total amount of genomic DNA that was inherited from one parent.

26. The method of claim 24 or claim 25, wherein the total amount of genomic DNA is about 3,560 cM.

27. The method of any one of claims 1-26, wherein each of the plurality of kinship windows comprises between 25 and 200 SNPs.

28. The method of any one of claims 1-27, wherein each of the plurality of kinship windows comprises between 75 and 125 SNPs.

29. The method of any one of claims 1-28, wherein each of the plurality of kinship windows comprises about 60 SNPs or 100 SNPs.

30. The method of any one of claims 1-27, wherein each of the plurality of kinship windows comprises a length of between 5 and 70 centimorgan (cM).

31. The method of any one of claims 1-27 and 30, wherein each of the plurality of kinship windows comprises a length of between 20 and 40 cM.

32. The method of any one of claims 1-27, 30, and 31, wherein each of the plurality of kinship windows comprises a length of about 20 cM.

33. The method of any one of claims 1-27 and 30, wherein each of the plurality of kinship windows comprises between 5 and 70 million base pairs.

34. The method of any one of claims 1-27, 30, and 33, wherein each of the plurality of kinship windows comprises between 20 and 40 million base pairs.

35. The method of any one of claims 1-27, 30, 33, and 34, wherein each of the plurality of kinship windows comprises about 20 million base pairs.

36. A method for performing DNA-based kinship analysis, comprising:
providing a nucleic acid sample;
amplifying the nucleic acid sample with a plurality of primers that specifically hybridize to a plurality of target sequences collectively comprising a plurality of single nucleotide polymorphisms (SNPs), thereby generating amplification products;
sequencing the amplification products;
determining the genotypes of the plurality of SNPs, thereby generating a DNA profile; and
calculating the degree of relationship of the DNA profile to a reference DNA profile, wherein the calculating comprises determining a chromosome-specific kinship value for each of two or more pairs of chromosomes.

37. The method of claim 36, wherein the two or more pairs of chromosomes comprises 10 or more, 11 or more, 12 or more, 13 or more, 14 or more, 15 or more, 16 or more, 17 or more, 18 or more, 19 or more, 20 or more, 21 or more, 22 or more, or 23 pairs of chromosomes.

38. The method of claim 36 or 37, wherein the two or more pairs of chromosomes comprises 22 pairs of chromosomes.

39. The method of any one of claims 36-38, wherein the calculating comprises determining a total number of shared SNPs between the DNA profile and the reference DNA profile.

40. The method of any one of claims 36-39, wherein the determining the chromosome specific kinship value is based on a comparison of the shared SNPs between the DNA profile and the reference DNA profile.

41. The method of claim 40, wherein the comparison comprises determining a total number of overlapping SNPs between the DNA profile and the reference DNA profile.

42. The method of any one of claims 36-41, wherein the determining the chromosome specific kinship value for each of the two or more pairs of chromosomes is performed using algorithms and processes of, associated with, or derived from, PC-Relate.

43. The method of any one of claims 36-42, wherein the calculating an overall CSKP score for the DNA profile in comparison to the reference DNA profile comprises the use of a random forest model and a chromosome specific kinship value for each of the two or more pairs of chromosomes.

44. The method of claim 43, wherein the generating a CSKP model comprises:
(a) calculating a mean and standard deviation of chromosome kinship for each chromosome contained within an unrelated sample training set, wherein the unrelated sample training set comprises samples from unrelated individuals;
(b) calculating a z-score for each chromosome kinship;
(c) calculating a log survival function on the z-score;
(d) calculating a sum of the log survival function for each of the two or more chromosomes;
(e) performing a logistic regression on the sum of the log survival function; and
(f) training a random forest model on overall kinship, log probability from logistic regression analysis, and total overlapping SNPs between samples.

45. The method of claim 44, wherein the calculating an overall CSKP score comprises:
(a) determining a chromosome specific kinship value for each of the two or more pairs of chromosomes and calculating a z-score for each chromosome specific kinship value based on the mean and standard deviation of chromosome kinship for the unrelated sample training set;
(b) calculating a log survival function value for the z-score of each chromosome specific kinship value and summing the log survival function values;
(c) calculating a log probability using the summed value of log survival function values; and
(d) determining an overall CSKP score using the random forest model based on the log probability, the total number of shared SNPs between the DNA profile and the reference DNA profile, and the overall kinship value.

46. The method of any one of claims 36-42, wherein the calculating an overall CSKP score for the DNA profile in comparison to the reference DNA profile comprises the use of a random forest model and a chromosome specific kinship value for each of the two or more pairs of chromosomes.

47. The method of claim 46, wherein the calculating an overall CSKP score comprises:
(a) determining a chromosome specific kinship value for each of the two or more pairs of chromosomes and calculating a z-score for each chromosome specific kinship value based on a mean and standard deviation of chromosome kinship for an unrelated sample training set,
wherein the mean and standard deviation of chromosome kinship for the unrelated sample training set were determined by a CSKP model comprising the steps of:
(i) calculating a mean and standard deviation of chromosome kinship for each chromosome contained within an unrelated sample training set, wherein the unrelated sample training set comprises samples from unrelated individuals;
(ii) calculating a z-score for each chromosome kinship;
(iii) calculating a log survival function on the z-score;
(iv) calculating a sum of the log survival function for each of the two or more chromosomes;
(v) performing a logistic regression on the sum of the log survival function; and
(vi) training a random forest model on overall kinship, log probability from logistic regression analysis, and total overlapping SNPs between samples; and
(b) calculating a log survival function value for the z-score of each chromosome specific kinship value and summing the log survival function values;
(c) calculating a log probability using the summed value of log survival function values; and
(d) determining the overall CSKP score using the random forest model based on the log probability, the total number of shared SNPs between the DNA profile and the reference DNA profile, and the overall kinship value.

48. The method of any one of claims 36-47, wherein the overall CSKP score for the DNA profile each reference DNA profile represents the relatedness of the DNA profile with the reference DNA profile.

49. The method of any one of claims 1-48, wherein the plurality of SNPs comprises between 5,000 and 50,000 SNPs.

50. The method of any one of claims 1-49, wherein the plurality of SNPs comprises between 5,000 and 15,000 SNPs.

51. The method of any one of claims 1-50, wherein the plurality of SNPs comprises between 9,000 and 11,000 SNPs.

52. The method of any one of claims 1-51, wherein the amplification is carried out in one or more multiplex PCR reactions

53. The method of any one of claims 1-52, wherein the sequencing is conducted using massively parallel sequencing (MPS).

54. The method of any one of claims 1-53, wherein the sequencing does not comprise whole genome sequencing (WGS).

55. The method of any one of claims 1-54, wherein the nucleic acid sample comprises genomic DNA.

56. The method of any one of claims 1-55, wherein the nucleic acid sample comprises one or more enzyme inhibitors.

57. The method of claim 56, wherein the one or more enzyme inhibitors comprise one or more inhibitors selected from the group consisting of hematin, humic acid, indigo, and tannic acid.

58. The method of any one of claims 1-57, wherein the nucleic acid sample comprises low-quality nucleic acid molecules and/or low quantity nucleic acid molecules.

59. The method of claim 58, wherein the low quality nucleic acid molecules are degraded genomic DNA and/or fragmented genomic DNA.

60. The method of any one of claims 1-59, wherein the nucleic acid sample is a forensic sample.

61. The method of any one of claims 1-60, wherein the nucleic acid sample is derived from a buccal swab, paper, fabric, or other substrate that is impregnated with saliva, blood, or other bodily fluid.

62. The method of any one of claims 1-61, wherein the nucleic acid sample comprises between or between about 50 pg and 100 ng of genomic DNA.

63. The method of any one of claims 1-62, wherein the nucleic acid sample comprises between or between about 100 pg and 5 ng of genomic DNA.

64. The method of any one of claims 1-63, wherein the nucleic acid sample comprises at or about 1 ng of genomic DNA.

65. The method of any one of claims 1-64, wherein the plurality of SNPs comprises kinship SNPs.

66. The method of any one of claims 1-65, wherein the plurality of SNPs comprises kinship SNPs, ancestry SNPs, identity SNPs, phenotype SNPs, X-SNPs, and Y-SNPs.

67. The method of any one of claims 1-66, wherein the plurality of SNPs comprises SNPs selected from one or more of the groups consisting of kinship SNPs, ancestry SNPs, identity SNPs, phenotype SNPs, X-SNPs, and Y-SNPs.

68. The method of any one of claims 1-67, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

69. The method of any one of claims 1-68, wherein the DNA profile in relation to the reference DNA profile is a relative of the first degree, second degree, third degree, fourth degree, fifth degree, sixth degree, or seventh degree.

70. The method of claim 69, wherein the DNA profile in relation to the reference DNA profile is a relative of the fourth degree or fifth degree.

71. The method of any one of claims 1-70, further comprising generating a family tree comprising the DNA profile in relation to the reference DNA profile.
